(19) United States
(12) Patent Application Publication
(10) Pub. No.: US 2011/0294730 A1
(43) Pub. Date: Dec. 1, 2011

(54) METHOD OF TREATING GLAUCOMA AND INTRAOCULAR HYPERTENSION

(76) Inventors: Totada R. Shantha, McDonough, GA (US); Erica Maya Shantha, McDonough, GA (US); Jessica Gowramma Shantha, McDonough, GA (US)

(21) Appl. No.: 12/790,964

(22) Filed: May 31, 2010

(51) Int. Cl. A61K 38/28; A61P 27/06

(52) U.S. Cl. 514/6.5; 514/20.8

(57) ABSTRACT

A safe and effective treatment for glaucoma for mammalian species comprises the steps of applying insulin and/or insulin like growth factors (IGF-1) to an eye. In addition to the insulin, another therapeutic agent may be applied to enhance the activity of the insulin. The therapeutic agent may be a pharmaceutical agent or a biochemical pharmaceutical agent. The therapeutic agents include prostaglandin analogs, topical beta-adrenergic receptor antagonists-β blockers, Alpha2-adrenergic agonists hair growth therapeutic agents, beta2-agonist action agents, parasympathomimetic miotic agents, carbonic anhydrase inhibitors, and Physostigmine. In another embodiment, a combination of at least two agents are applied to the eye. To enhance the effect of the insulin, uptake facilitators may be used. Additionally, an antibacterial agent may be applied to control bacterial infection.

### FIELD OF THE INVENTION

The invention relates to method for treating ocular hypertension, glaucoma with retinal damage and loss of vision in human and mammals.

### BACKGROUND OF THE INVENTION

Glaucoma is a disease characterize by the increase of intraocular pressure (IOP) due to varied pathogenesis with damage to the retina and atrophy of the optic nerve resulting in the abnormal visual field, with reduced visual acuity. The optic nerve does not recover once atrophy occurs, even if the raised IOP is corrected. Elevated intraocular pressure (IOP) is a very significant risk factor for the development of the majority of common forms of glaucoma (Sommer A, et al., “Relationship Between Intraocular Pressure and Primary Open Angle Glaucoma Among White and Black Americans,” Arch. Ophthalmol, 109:1090-1095 (1991)). Ocular hypertension with the absence of visual field defects, may lead to development of glaucoma over a long time, also has a similar risk.

Some of the Risk Factors for developing glaucoma are: High Intraocular (Eye) Pressure (IOP), Old age, family history of glaucoma, Race (Blacks), Suspicious optic nerve appearance (cupping >50% or asymmetry), Central corneal thickness less than 555 microns (0.5 mm), High Myopia (near sightedness), Diabetes, Hypertension, Eye Injury or Surgery, History of steroid use, Migraine headache and peripheral vasospasm, Sleep-related breathing disorder and male Gender. Primary open angle glaucoma occurs in approximately 4% of diabetics compared to 1.8% of the general population.

The blindness is the second most feared affliction, next only to cancer. Glaucoma which can result in blindness and affects approximately five percent of population older than 65 years and fourteen percent of those older than 80 years. It is the leading cause of irreparable permanent blindness all over the world. An estimated 70 million people have glaucoma worldwide. Nearly 7 million are bilaterally blind from this disease due to damage to the retina. In the United States alone, about 3-4 million people suffer from glaucoma and is the third common reason for adults to visit a physician in US. Many people who have the disease do not even know that that they have this serious blinding affliction of the eye. Animals can also be affected by this condition. It is estimated there are 65 million dogs in North America, of which approximately 1.3 million will develop glaucoma, if untreated they go blind.

The incidence of glaucoma and blindness are different among different races, and the blindness from glaucoma is four to eight times more common in African-Americans than in Caucasian Americans. Overall, glaucoma is more severe in black patients (Higginbotham et al., Baseline Characteristics of Black and White Patients, Ophthalmology 105(7):1137-1145, 1996. Higginbotham et al., The advanced glaucoma intervention study (AGIS):3. Leske, M. C. et al., Arch. Ophthalmol. 112:821-829, 1994. Tielsch J M, Sommer A, Katz J, Royall R M, Quigley H A, Javitt J. Racial variations in the prevalence of primary open-angle glaucoma. The Baltimore Eye Survey. JAMA 1991; 266:369-74). It appears that the risk for developing glaucoma may be inherited.

Glaucoma is categorized into: 1. congenital (developmental) glaucoma, 2. Primary glaucoma and 3. Secondary glaucoma. Patients with congenital (developmental) glaucoma are born with growth deficiency of the iridocomeal angle, resulting in obstruction to the aqueous outflow resulting in this type of glaucoma. Secondary glaucoma arises as a result of inflammation or injury and is caused by such as uveitis or ocular trauma as well as hemorrhage due to diabetes, long-term use of steroid hormones for the treatment of other diseases, and the like. Primary glaucoma is a generic name of glaucoma's of many types with unclear etiology; most common form of glaucoma, with a high incidence among middle-aged and elderly persons. Primary glaucoma and secondary glaucoma are further subdivided into two types, open-angle glaucoma and closure-angle glaucoma, depending on the blockage of the aqueous outflow. While many patients develop normal tension glaucoma in the absence of elevated IOP, the primary aim of glaucoma treatment is to lower the IOP and prevent further retinal damage.

Histology, Pathophysiology and Paramacology of the Trabecular Mesh Work™ and Aqueous Humor Dynamics in Relation to Glaucoma as it Related to our Invention

To understand the glaucoma and its treatment using our invention, the knowledge of the histology of the eye related to aqueous humor production and its final exit from the anterior and posterior chambers of the eye (FIGS. 1-5) is useful. The lens of the human eye is suspended in the eye by zonule ligament fibers and is positioned against the back of the iris and divides the anterior part of the eye ball in to 3 compartments (FIGS. 1,3-5). They are: 1. Anterior Chamber between the iris and the cornea; 2. Posterior chamber between the iris, zonule fibers and the lens 3. And the vitreous chamber is between the lens and the retina. The anterior chamber, posterior chamber and between the zonule ligament fibers, or a serrate, capillary space behind the lens are filled with aqueous humor. The vitreous chamber is filled with a viscous gelatinous material whose role is not established in glaucoma.

With advancing age, the lens becomes less compressible and is rigid compared to a 10 year old persons' lens which is malleable who have flat iris, and changes its shape and adjust its focus from six inches to infinity. By age 55 years the lens enlarges, moves forwards, is less malleable and becomes rigid. This makes the anterior chamber of the eye shallow with elevated intraocular pressure as age advances due to forward movement of the iris obstructing the free flow of the aqueous humor through its exits. It is estimated that the anterior chamber becomes ninety-eight percent shallower in the 80 years old compared to the 10 year children who have flat iris with deep anterior chambers.

Aqueous humor is secreted by the ciliary process behind the iris from where it flows to the posterior and anterior chambers (FIGS. 1-5). The aqueous humor in the anterior chamber passes through the trabecular meshwork™ to the Canal of Schlemm; and to the uveoscleral and conrneoscleral trabecular meshwork finally to the eye venous system, suprachoroidal and interchoridal spaces. Any factor that compromises the ability of fluid to drain through the trabecular meshwork and the Canal of Schlemm can result in IOP elevation in the anterior chamber. With advancing age, increased intraocular pressure (IOP) is necessary to force aqueous through the filtering tissue; when the pressure rises above normal, glaucoma results. By the age 20 less than 0.5% of patients have elevated IOP, and by the age 80 years over 4% of patients have glaucoma due to aqueous humor drainage passages become clogged, narrowed and obstructed over time.

The eye is surrounded and protected by the thick connective tissue sclera; which is the continuation of the Dural covering the optic nerve (FIGS. 1, 7). The cornea is front of the eye which is a clear window letting light in to retina. The iris is the colored part of the eye and controls the amount of light that can enter the eye by automatically opening and closing the pupil (the opening of the eye). The lens is behind the pupil focuses light onto the retina. Retinal nerve fibers and blood vessels gather to form the optic nerve which carries images to the occipital brain. The place where the optic nerve leaves the eye is called the optic disc which can become cup shaped due to elevated IOP. The front part of the eyeball is filled with aqueous humor secreted continuously by ciliary processes into the posterior chamber by the ciliary body, especially by the non-pigmented epithelium of the ciliary body. Aqueous humor is produced (secreted) and removed from the eye at a constant rate about 5 ml per day to maintain a constant pressure in the anterior chamber of the eye. While pressure in the eye varies throughout the day, the normal pressure within the eye is 10 mm Hg to 21 mm Hg. The ionic composition of aqueous humor is similar to the blood plasma but has less protein.

The aqueous humour is a thick watery substance located between the lens and the cornea. The anterior segment is the front third of the eye that includes the structures in front of the vitreous humour; the cornea, iris, ciliary body, and lens. Within the anterior segment are two fluid-filled spaces divided by the iris plane: Aqueous humour fills these spaces within the anterior segment to provide nutrients to the lens and corneal endothelium, and its pressure maintains the convex shape of the cornea and shape of the eye globe. In a healthy eye, the aqueous humour does not mix with the gel-like vitreous humour because of the lens and it's Zonule of Zinn between the two. Its main function is to provide diopteric power to the cornea besides maintaining the intraocular pressure; inflates the globe of the eye; provides nutrition (e.g. amino acids and glucose) for the avascular ocular tissues (posterior surface of the cornea, trabecular meshwork, lens and the anterior surface of the vitreous humor). It carries away waste products from metabolism of the above avascular ocular tissues; may serve to transport ascorbate in the anterior segment to act as an anti-oxidant agent; Presence of immune globulins indicate a role in immune response to defend against pathogens.

Since the aqueous humor is continuously produced; it must be proportionately drained continuously also. The fluid drains through an area called the drainage angle located where the iris and cornea meet to form the trabecular meshwork (the valve that regulates pressure within the eye) and to a channel called the Schlemm's canal; also known as canal of Schlemm or the scleral venous sinus (FIGS. 3, 4). It is a circular channel in the eye that collects aqueous humor from the anterior chamber and delivers it into the bloodstream. Finally aqueous humor exits the eyeball it drains into Schlemm's canal by one of two ways: directly, via aqueous vein to the episcleral vein, or indirectly, via collector channels to the episcleral vein by intrascleral plexus and eventually into the systemic veins of the orbit. The aqueous humor draining through the uveal and corneoscleral meshwork drains between the ciliary muscles, then to the choroid and choroidal plexus of blood vessels through supra and inter choroidal spaces and lymphatic channels.

When there is an obstruction to the drainage, the fluid builds up in the eye (called intraocular pressure or IOP) and presses against the optic nerve, resulting in glaucoma and vision changes. This increased aqueous humor fluid pressure as seen in ocular hypertension pushes the optic nerve back into a “cupped” or concave shape. If the intraocular pressure remains too high for long periods of time, the extra pressure damages parts of the optic nerve affecting the vision due to apoptosis of the ganglion cells of the retina. Glaucoma, more often than not is bilateral and the extra fluid pressure first begins to build up in one eye. There are many types of glaucoma as described below; but the two most common types are open-angle and closed-angle (angle-closure) glaucoma.

The trabecular mesh work™ plays an important role in drainage of aqueous humor and development of glaucoma. It has been delineated and described by Artur Llobet, Xavier Gasull and Arcadi Gual. Understanding Trabecular Meshwork Physiology: A Key to the Control of Intraocular Pressure? Physiological Sciences, 2003 Vol. 18, No. 5, 205-209; which is incorporated in here in its entirety. Trabecular meshwork contains endothelium-lined spaces with intertrabecular spaces similar to arachnoid villi (which drain CSF when there is elevated pressure) through which passes the aqueous humor to Schlemm's canal, uveal mesh work and corneoscleral meshwork. It was shown that these endothelial cells are nothing but the continuation of choroidal cells, which are in turn continuation of Pia-arachnoid matter from the optic nerve and CNS (FIGS. 1, 7). The trabecular meshwork is akin to arachnoid villi in the brain and spinal cord which play an important role in exit of CSF from subarachnoid spaces whenever the CSF fluid pressure rises. In the same manner trabecular meshwork plays a role in the exit of aqueous humor in the eye as the aqueous humor pressure builds up. Structurally, both have similar histological features with intercellular spaces for exit of fluids. Trabecular meshwork is nothing but the arachnoid villi in the anterior chamber of the eyes (FIG. 1). (Shantha T R and Bourne G H: Histological and Histochemical studies of the choroid of the eye and its relations to the pia-arachnoid mater of the central nervous system and Perineural epithelium of the peripheral nervous system. Acta Anat 61:379-398 (1965). Shantha T R and Bourne G H: Arachnoid villi in the optic nerve of man and monkey. Experimental Eye Research, 3:31-35 (1964). Shantha T R and Evans J A: Arachnoid Villi in the Spinal Cord, and Their Relationship to Epidural Anesthesia. Anesthesiology 37:543-557, 1972). It is important to note that there is a rich network of arteriovenous system supplying and draining the canal of Schlemm, sub-conjunctival space at the edge of the cornea and sclera, episcleral and interchoridal spaces, ciliary body, iris and ciliary muscles which play an important role in delivery of therapeutic agents (FIGS. 5, 5A) to these histological structures which play an important role in glaucoma production and therapy.

Trabecular meshwork (FIGS. 1-5): which plays an important role in draining of aqueous humor in healthy and raising the IOP in pathological state, is divided into:

1. The uveoscleral or nonconventional Inner uveal meshwork pathway—through which the aqueous humor exits by diffusion through intercellular spaces among ciliary and base of the iris muscle fibers. These histological pathways are the target of specific anti glaucoma therapeutic agents (latanoprost, a prostaglandin F₂* analog) that increase the functionality of this route. Closest to the anterior chamber angle, faces the anterior chamber; contains thin cord-like trabeculae, orientated predominantly in a radial fashion, enclosing trabecular spaces larger than the corneoscleral meshwork and said to participate 5-10% of aqueous humor outflow.

2. Corneoscleral meshwork—is in contact with the cornea and the sclera and continuous with the cribriform mesh work of Schlemm's canal. Corneoscleral meshwork—Contains a large amount of elastin, arranged as a series of thin, flat, perforated sheets arranged in a laminar pattern; considered to be the ciliary muscle tendon (Sampaolesi R, Sampaolesi J R, Zárate G (2009). “Ocular Embryology with Special Reference to Chamber Angle Development” (chapter 8). The Glaucomas—Pediatric Glaucomas (volume 1) pp. 61-69 Springer Berlin Heidelberg). When the ciliary muscle contracts, it opens up the conrenoscleral T M and allow more and rapid flow of aqueous humor, thus reducing the IOP. It is important to note that the choirdal lamellae extend all the way to the trabecular meshwork and iris, hence the continuation of the pia-arachnoid from the optic never. The aqueous humor from the trabecular meshwork drains into the suprachoroidal space (supra ciliary space) which is nothing but the intra choroidal space then to the artio-venous system and sub-arachnored space of the optic nerve specially in lower animals such as rabbits (Shantha, T R and Bourne G H: Histological and Histochemical studies of the choroid of the eye and its relations to the pia-arachnoid mater of the central nervous system and Perineural epithelium of the peripheral nervous system. Acta Anat 61:379-398, 1965. Nilsson S F. The uveoscleral outflow routes. Eye (Lond). 1997; 11 (Pt 2):149-54). The spaces between the choroid are lined by endothelium (a continuation of Pia-arachnoid from the optic nerve), and open freely into the peri choroidal lymph space, which in turn, communicates with the peri scleral space by the perforations in the sclera through which the vessels and nerves are transmitted. Some of the aqueous humor from the suprachoroidal space escapes through these lymphatics, though slowly.

There are no epithelial barriers between the anterior chamber and the ciliary muscles. Hence the aqueous humour freely passes between the ciliary muscle bundles into the suprachoroidal (supraciliary) and inter choroidal spaces and, from which it is drained through the sclera and choroidal BV. This uveoscleral outflow of aqueous humour accounts for 40-60% of the total outflow in monkeys. Direct measurements in human eyes have suggested that less than 15% is drained by the uveoscleral routes. However, indirect calculations have given a value of about 35% in young adults and 3% in elderly persons (>60 years). The uveoscleral outflow is decreased by contraction (pilocarpine) and increased by relaxation (atropine) of the ciliary muscle. Thus, changing the tone of the ciliary muscle may redistribute aqueous humour flow between the conventional and uveoscleral outflow routes. Prostaglandins decrease the intraocular pressure by increasing the uveoscleral outflow. Two mechanisms seem to contribute to this effect: relaxation of the ciliary muscle and changes in extracellular matrix, causing decreased resistance in the uveoscleral outflow routes (Nillson IBID).

3. Juxtacanalicular tissue or cribriform meshwork-lies immediately adjacent to Schlemm's canal, composed of connective tissue ground substance full of glycoaminoglycans and glycoproteins. We believe, in diabetes, due to glycation, this substance increases many folds contributing to obstruction to free flow of aqueous humor resulting in the higher percentage of glaucoma in diabetics. This thin strip of tissue is covered by a monolayer of endothelial cells which form the wall of Schlemm's canal. Electron microscopy studies indicate that the aqueous humor crosses the inner wall endothelium of Schlemm's canal by two different mechanisms: a paracellular route through the junctions formed between the endothelial cells (Epstein D L and Rohen J W. Morphology of the trabecular meshwork and inner-wall endothelium after cationized ferritin perfusion in the monkey eye. Invest Ophthalmol Vis Sci 32: 160-171, 1991); and a transcellular pathway through intracellular pores of the same cells (Johnson M and Erickson K. Aqueous humor and the dynamics of its flow. In: Principles and practice of ophthalmology, edited by Albert D M and Jakobiec F A. Philadelphia: Saunders, 2000, p. 2577-2595). However, the functional importance of each of these two pathways is still tentative.

Besides these above routes; aqueous humor exits through other structures which may take part in the aqueous humor absorption path. The studies by Shantha and Bourne have shown the presence of intercellular pores (FIG. 4) between the corneal endothelial cells distributed in a dotted fashion between cell junctions. These pores are more numerous and larger as the corneal endothelium approaches the trabecular meshwork and corneoscleral junction (Shantha T R and Bourne G H: Some observations on the corneal endothelium. Acta Ophthalmologica 41: 683-688 (1963) (FIG. 4). Some of the aqueous humor outflows via these intercellular spaces of the corneal endothelium (FIG. 4) to the corneal, scleral, and descemets membrane of the cornea to the corneal epithelium, canal of Schlemm and sinus—episcleral venous system (FIGS. 3, 5A).

Histochemical studies of oxidative, dephosphorylating enzyme and β-glucuronidase have shown that these trabecular meshwork endothelial cells showed a strong positive activity for succinic dehydrogenase and monoamine oxidase; but they were moderately positive for the beta glucuronidase and only mildly positive for cytochrome oxidase. Among posphorylating groups of enzymes, strong positive activity was observed for the alkaline phosphatase, acid phosphatase, glucose-6-phosphatase and ATPase; moderate positive activity was observed for inosine diphosphatase, uridine triphosphatase, thiamine pyrophosphatase and pyridoxal phosphatase; and mild positive activity was observed for 5′ nucleotidase and creatine phosphatase. In the thiamine pyrophosphatase preparations we observed a large dense mass of black-staining material vesicular in shape in the cytoplasm of these meshwork endothelial cells. The rest of the cytoplasm was diffusely stained by this technique. The corneal endothelium also showed the same type of distribution of these enzymes as the meshwork cells. All these results have indicated that the meshwork cells are metabolically highly active in view of their high histochemicallly demonstrated enzyme activity with high ATP energy production. Though the function of these highly enzymatically active cells is not known, various suggestions are made including a secretory, transporting and synthesizing activity in view of the available experimental evidence. Further experimental evidence is awaited to know their exact role they play in drainage of aqueous humor and pathological changes they undergo in glaucoma (Shantha T. R. and Bourne G. H: Histochemical Studies On The Distribution Of Oxidative And Dephosphorylating Groups Of Enzymes In The Meshwork Cells Of The Anterior Chamber Angle Of The Eye. American Journal of Ophthalmology. Vol. 60, No. 1, July, 1965 pp 49-55).

Decline in the Cell Population of the Trabecular Meshwork of Aqueous Humor Outflow System and its Relation to Glaucoma Therapy and the Present Invention

The cell population of the outflow system (meshwork) of the normal eye reduced with escalating ever-increasing age, (Alvarado J, Murphy C, Polansky J, Juster R. Age related changes in trabecular meshwork cellularity. Invest Ophthalmol Vis Sci. 1981; 21:714-727. Alvarado J, Murphy C, Juster R. Trabecular meshwork cellularity in primary open-angle glaucoma and non glaucomatous normals. Ophthalmology. 1984; 91:564-579. Grierson I, Howes R C. Age-related depletion of the cell population in the human trabecular meshwork. Eye. 1987; 1:204 -210). This cell loss may precipitate some of the structural alterations that occur during the aging process such as trabecular thickening and trabecular fusion contributing to glaucoma (Grierson I, Wang D, McMenamin P G, Lee W R. The effects of age and anti-glaucoma drugs on the meshwork cell population. Res Clin Forums. 1982; 4:69 -92).

The age-related cell loss is even more pronounced in patients with primary open-angle glaucoma (POAG) than in age-matched regular normal eyes without glaucoma. It has been suggested that excessive cell loss is an early, and perhaps the primary, pathologic event in the outflow system in POAG (Grierson I. What is open angle glaucoma? Eye. 1987; 1:15-28). The mechanism of cell loss and the environmental factors contributing to it are not known. The meshwork cell loss may be brought about by cell death caused by mechanical stress (Grierson I. What is open angle glaucoma? Eye. 1987; 1:15-28) or by noxious insult such as free radical attack and age related glycation.

It has been suggested a further mechanism for meshwork cell depletion in health and disease namely detachment from the trabeculae and migration from the outflow system, (Grierson I, Calthorpe C M. Characteristics of meshwork cells and age changes in the outflow system of the eye: their relevance to primary open angle glaucoma. In: Mills K B, ed. Glaucoma. Oxford: Persimmon Press; 1989: 12-31. 10). Meshwork cells, provoked by a variety of stimuli, become “activated,” detach from their neighboring cells on the trabeculae, undergo shape changes and then migrate to Schlemm's canal and trabecular meshwork and then pass through the endothelium into the lumen of the canal into the venous drainage. The “activation” process is associated with excessive phagocytosis, inflammation, injury, or a combination of all three processes (Rohen J W, Van Der Zypen E. The phagocytic activity of the trabecular meshwork endothelium. Graefes Arch Clin Exp Ophthalmol. 1968; 175:251-266).

It is probable in these circumstances that the aqueous fluid, which bathes the meshwork cells, contains motogenic factors that stimulate meshwork cell migration. It is also possible that normal aqueous humor and the aqueous humor from POAG patients contain motogens. If so, then the steady attrition of meshwork cells might be caused by a slow version of the process seen after inflammatory and particulate insult. It is also possible, that due to unknown insult or just due to aging, the stickiness of these endothelial cells also reduced, making them easily detached from the anchor and start moving in these glaucoma patients. Electron microscopic studies show meshwork cells partially detached from the trabeculae in the normal aging meshwork and in trabeculectomy specimens from POAGs (Grierson I. What is open angle glaucoma? Eye. 1987; 1:15-28).

Migration is likely to be a difficult event to study in vivo because cell loss has been calculated to be at most only in the region of 20 cells per day. In addition, any chemo attractants found in aqueous humor are likely to be there in very small quantities. The microchemoattraction chamber assay is a sensitive procedure that serves both to quantify and to analyze migration in vitro. In the case of meshwork cell migration it also acts to amplify in vitro the small migratory changes that may be stimulated by aqueous fluid in vivo. The corneal endothelium, scleral fibroblasts, the glycoprotein's, fibronectin (Fn) and laminin; in addition, platelet-derived growth factor are a highly potent migratory stimulant, whereas EGF and bFGF show no activity at all; others lie somewhere in between. When bovine aqueous was used as a stimulant the bovine meshwork cells responded to it as well as they did to optimal concentrations of soluble fibronectin (sFn) (Hogg P, Calthorpe C M, Ward S, Grierson I. The migration of cultured bovine trabecular meshwork cells to aqueous humor and constituents. Invest Ophthalmol Vis Sci. 1995; 36:2449-2460). If the meshwork cell migration is caused by chemo attractants in aqueous explains part of the cell loss in aging, then it follows that the excessive cell loss associated with POAG may be due to glaucomatous aqueous being a more effective chemoattractant than nonglaucomatous aqueous.

Aqueous humor contains potentially powerful chemoattractants for trabecular meshwork cells. The activity of one of these constituents, fibronectin, has been accounted for by the study of Hogg et al. Glaucomatous aqueous appears to be as good and in some cases a better migratory stimulant than nonglaucomatous aqueous in vitro. The migratory evidence points to a trend that may help to explain cell loss in the aging meshwork and possibly some of the extra loss in primary open-angle glaucoma (Penny Hogg, Mary Calthorpe, Mark Batterbury, and Ian Grierson. Aqueous Humor Stimulates the Migration of Human Trabecular Meshwork Cells In Vitro. Invest Ophthalmol Vis Sci. 2000; 41:1091-1098). Our invention can increase the cell population; because insulin is mitotic (induces cell division) inducer; and alleviated glaucoma related to this pathophysiology of trabecular meshwork cell loss and restores retinal function.

In a healthy eye, the aqueous humour does not mix with the gel-like vitreous humour because of the lens and it's Zonule of Zinn between the two structures. There is likely a thin capillary aqueous flow permeates between the suspensory ligament, behind the lens, the retinal edge (or a serrata) and vitreous humor. It is not known how much aqueous humor exits or absorbed through these routes and their role in glaucoma.

Although the anatomic organization of the TM indicate the unique regulatory properties of this structure in relation to the outflow of aqueous humor in maintaining IOP preventing the glaucoma development, the ciliary muscle ligament insertions to the TM modulate the permeability of this tissue to aqueous humor (Rohen J W, Futa R, and Lutjen-Drecoll E. The fine structure of the cribriform meshwork in normal and glaucomatous eyes as seen in tangential sections. Invest Ophthalmol Vis Sci 21: 574-585, 1981). When the ciliary muscle contracts, its insertions widen the intercellular spaces in the TM and the permeability of the tissue increases; simultaneously, uveoscleral outflow decreases. When the ciliary muscle relaxes, the intercellular spaces of the TM become narrower and the trabecular outflow is reduced. Correspondingly, the uveoscleral outflow is increased (FIGS. 1-5). Therefore, the aqueous humor outflow is regulated between the trabecular and uveoscleral pathways depending on the tone of the ciliary muscle (Wiederholt et al. in Bill A. Blood circulation and fluid dynamics in the eye. Physiol Rev 55: 383-417, 1975).

Finally, we should also be aware that the TM has autonomic and sensory innervations, which may release different neurotransmitters to modulate trabecular meshwork permeability and aqueous humor flow (Ruskell G L. The source of nerve fibers of the trabeculae and adjacent structures in monkey eyes. Exp Eye Res 23: 449-459, 1976). Specific receptors for neurotransmitters and neuropeptides, including epinephrine, acetylcholine, and neuropeptide Y, have been identified in TM cells, indicating that they can detect the activity of sensory and autonomic fibers innervating the TM which can also play a role in the flow of aqueous humor and genesis of glaucoma. Trabecular meshwork cells are enzymatically rich indicating their active participation in neurotransmitters and aqueous humor flow (TRS. American Journal of Ophthalmology. Vol. 60, 1965, pp 49-55).

There is a long list of vasoactive peptides and growth factors (e.g., endothelin-1, bradykinin, etc.) trigger intracellular signaling mechanisms in TM cells. All of these factors are active at very low concentrations (i.e., in the nanomolar range), and it is likely that tissues surrounding anterior and posterior chambers secrete these substances, which might control TM function in a paracrine manner. For example, it has been documented that the non-pigmented cells of the ciliary body secrete substances to the aqueous humor such as atrial natriuretic peptide, endothelin-1, or galanin that could activate their specific membrane receptors in TM cells (Coca-Prados M, Escribano J, and Ortego J. Differential gene expression in the human ciliary epithelium. Prog Retin Eye Res 18: 403-429, 1999). Drugs mimicking parasympathetic nerve stimulation (pilocarpine-a muscarinic agonist), contracts the ciliary muscle, increase aqueous humor drained through the conventional trabecular meshwork outflow pathway. Drugs like epinephrine relax the muscle through activation of β₂-adrenoceptors but, contrary to expectations, they increased trabecular outflow and thereby are used in anti-glaucoma therapy. Along the same line, it has been shown that when the ciliary muscle is excised from the TM, bradykinin and serotonin decrease aqueous humor outflow.

So, in addition to the ciliary muscle, what are the other modulators of trabecular meshwork permeability? Experiments have shown that the TM is a contractile tissue with properties similar to smooth muscle. It contracts when exposed to muscarinic agonists, α₁-adrenergic agonists, and endothelin-1, and it relaxes when β₂-adrenergic agonists, L-type Ca²⁺ channel blockers, or nitric oxide donors are applied. For example, TM contractility is linked to Rho kinase A, which can be regulated by PKC isoforms and does not require Ca²⁺ for its activation (Rho kinaseA inhibits myosin phosphatase, resulting in an accumulation of phosphorylated myosin light chain, which is then capable of interacting with actin to produce contraction). The contraction of the TM cells decreases the permeability of the TM because the size of the intercellular spaces is reduced. Similarly, when TM cells relax, the opposite effect appears and the permeability of the tissue increases.

There is a large body of proof suggesting that glucocorticoids are involved in the generation of ocular hypertension and glaucoma (Clark, A F, “Steroids, Ocular Hypertension and Glaucoma” J. Glaucoma 4:354-369, 1995). Several investigators have shown that the human trabecular meshwork™ contains the standard glucocorticoid receptor (hGRa) and its isoforms, hGRz (SEQ ID NO.3) (Weinreb, R N, Bloom, E, Baxter, J D, Avarado, J, Lan, N, O'Donnell, J, Polansky, J R, “Detection of Glucocortkoid Receptors in Cultured Human Trabecular Cells” INVEST. OPHTHALMOL. VIS. Sd. 21(3):403-407 (1981). Hernandez, M R, Wenk, E J, Weinstein, B I, Abumohor, P, Podos, S M, Dunn, M W, Southren, A L, “Glucocorticoid Target Cells in Human Outflow Pathway:Autopsy and Surgical Specimens” INVEST. OPHTHALMOL. VIS. SCI. 24:1612-1616, 1983). They may play a role in IOP regulation and development of glaucoma.

Up till now, many aspects of the regulation of aqueous humor production and outflow remain still vague and tentative. This fact is substantiated by a number of different classes of drugs used for the treatment of glaucoma and the definite glaucoma therapy is still in the horizon. The drugs most commonly used to treat open-angle glaucoma either decrease the production of aqueous humor in the ciliary processes or increase the uveoscleral and TM outflow or both. Drugs acting directly on the TM have not yet been developed, possibly because the mechanisms governing the function of this tissue are just beginning to be explained which may result in development of future anti glaucoma therapy directed toward the increase of trabecular outflow, decrease the loss of trabecular meshwork cells; at the same time reduce the pathological processes involved in retinal pathology. Our intention is directed towards these goals.

Types of Glaucoma that are Considered for Intervention Using our Inventive Method in Addition

Again, the Glaucoma is defined as a group of eye diseases characterized by increased intraocular aqueous humor pressure (IOP) with damage to the optic nerve (the nerve that sends the signal of images created by the eye to the brain). Glaucoma and IOP elevation are often erroneously considered synonymous. In actuality, glaucoma is characterized by damage in the optic nerve that results in visual field loss regardless of the IOP level. IOP is within the statistically “normal” range (less than 21 mm Hg) in 16% of the patients with glaucoma. This condition is termed “normal-tension glaucoma” On the other hand many patients with elevated IOP show no evidence of optic nerve damage or visual field loss. This condition is known as “ocular hypertension.” Much debate exists as to when glaucoma therapy should be initiated in patients with mild ocular hypertension alone with retinal changes. As the elevated IOP goes untreated, the damage to the optic nerve results in loss of peripheral and then central vision. The average IOP in a normal population is 14-16 millimeters of mercury (mmHg). In a normal population pressures up to 20 mmHg normal. A pressure of 22 is considered to be abnormal. However, not all patients with elevated IOP develop glaucoma-related retinal eye damage. What causes one person to develop damage while another does not, is under study.

There are different types of glaucoma due to different etiological factors and pathology, resulting in varied therapeutic agents to treat the condition. It could be primary without known etiology or secondary where there is contributing cause. The following list is modified from Wikipedia on Glaucoma, which is modified and incorporated herein. Our invention can be used in almost every type of glaucoma and rise in IOP compare to any anti glaucoma therapeutic agents and to prevent or treat retinal damage. The most common forms of glaucoma are as follows:

Primary Glaucoma:

Primary open-angle glaucoma, (85%) also known as chronic open-angle glaucoma, chronic simple glaucoma, glaucoma simplex

Primary angle-closure glaucoma also named as primary closed-angle glaucoma, narrow-angle glaucoma, pupil-block glaucoma, acute congestive glaucoma

Angle-Closure Glaucoma;

Acute angle-closure glaucoma or Acute Glaucoma

Chronic angle-closure glaucoma

Intermittent angle-closure glaucoma

Superimposed on chronic open-angle closure glaucoma (“combined mechanism” uncommon)

High-tension glaucoma with very high IOP

Normal Tension Glaucoma: Low-tension glaucoma with low or normal IOP

Variants of primary glaucoma

Pigmentary glaucoma

Exfoliation Syndrome

Exfoliation glaucoma, also known as pseudoexfoliative glaucoma or glaucoma capsulare

Secondary Glaucoma

Inflammatory glaucoma, Uveitis related glaucoma, Fuchs heterochromic iridocyclitis

Glaucoma due to Subluxation of lens, Phacogenic glaucoma and Angle-closure glaucoma with mature cataract, Phacoanaphylactic glaucoma secondary to rupture of lens capsule or Phacolytic glaucoma due to phacotoxic meshwork blockage; Postsurgical and Aphakic pupillary block glaucoma

Glaucoma secondary to intraocular hemorrhage and Hyphema (damage to the eye result in bleeding in to the anterior chamber); Hemolytic glaucoma (erythroclastic glaucoma)

Trauma-Related Glaucoma: blow to the eye, chemical burn, or penetrating injury may lead to the development of acute or chronic glaucoma due to a mechanical disturbance or physical change within the TM drainage system and Angle recession glaucoma can result from the traumatic depression of the anterior chamber angle

Ciliary block glaucoma

Neovascular glaucoma

Drug-induced glaucoma; due to corticosteroid and Postoperative ocular hypertension from use of alpha chymotrypsin.

Miscellaneous etiology: intraocular tumors, retinal detachments, essential iris atrophy Herpes Zoster ophthalmicus, and Toxic Glaucoma

Developmental Glaucoma

Primary congenital glaucoma

Infantile glaucoma

Glaucoma associated with hereditary of familial diseases

It is not the place to discuss all the glaucoma except the most common seen in practice which clinicians wants to treat with our invention described herein.

Primary Open-Angle Glaucoma (POAG)

Just about, one percent of Americans and 85% of all the glaucoma have this form of glaucoma; occurs mainly in the over 50 age group; often Painless with no symptoms due to slowly rising intraocular pressure (IOP); may be even undetected. By the time the vision is impaired, the visual loss is irreversible because once the nerve cells are dead; nothing can restore them. In POAG, there is no visible abnormality of the trabecular meshwork™ and is said to be due to inability of the cells in the trabecular meshwork to carry out their normal function, or there may be fewer cells present due to aging.

Increased IOP ultimately destroy the optic nerve ganglion and receptors cells with blind spots begin to form in the field of vision mainly in the outer sides of the field of vision; and in the later stages, the central vision is affected. Studies do suggested that the meshwork cell loss, which is associated with aging of the outflow system, and which is more marked in POAG, may result in part from meshwork cells being stimulated to migrate away from the trabeculae (Alvarado, J, Murphy, C, Juster, R. (1984) Trabecular meshwork cellularity in primary open-angle glaucoma and non glaucomatous normal's. Ophthalmology 91, 564-579. Grierson, I, Hogg, P. (1995) as described above. The anterior chambers become shallower by 30% or more over time due to the enlargement of the lens with its forward movement. Consider Lens exchange for this condition in the aged to alleviate glaucoma as one of the therapy if no other etiology is elicited.

Normal Tension Glaucoma (NTG)

Normal-tension glaucoma is also known as low-tension glaucoma, is characterized by progressive optic nerve damage and visual field loss with normal IOP and may account for as many as one-third of the cases of open-angle glaucoma in US. NTG is thought to be in part, to poor blood flow to the optic nerve, which leads to death of the ganglion cells which carry impulses from the retina to the brain. A pressure lower than normal is necessary to prevent further visual loss. Research in the field of optic nerve blood flow and its role in glaucoma is a source of much excitement at the present time and, hopefully, will lead to new methods of treating this type of eye disorder. Since the best therapy for normal-tension glaucoma is unknown, attention is given to a study known as the International Collaborative Low Tension Glaucoma Protocol and our invention may prevent the progression of this disease by improving the retinal physiology to normalcy due to insulin trophic effects.

Angle-Closure Glaucoma (ACG)

Angle-closure glaucoma affects 500,000 people in the US. Several members of a family may afflicted due to possible inheritance. It is common in Asian descendents and those who are far-sighted. In ACG, the anterior chamber is smaller than average and the TM is situated at 45 degrees angle formed where the cornea and the iris meet. The narrower the angle, the closer the iris is to the trabecular meshwork predisposing to this condition. With advancing age, the lens grows larger and thicker antero-posteriorly. The ability of aqueous generated by the ciliary epithelium process has to pass between the iris and lens on its way to the anterior chamber becomes decreased. This causes fluid pressure to build up behind the iris, further narrowing the TM angle. If the pressure becomes sufficiently high, the iris is forced against the trabecular meshwork, blocking drainage, resulting in ACG.

Narrow Angle Glaucoma occurs when the peripheral iris contacts the inner corneal wall. The normal passage of aqueous is blocked in its movement from the anterior chamber to the trabecular meshwork on with abrupt pressure rise IOP creates an acute glaucoma attack. When Conventional treatment with eye fail, the next step is to create a hole in the peripheral iris. Iridectomy by surgery or an iridotomy by laser significantly reduces the elevated pressure by creating an alternative passage for aqueous which move through the peripheral iris hole into the trabecular meshwork.

Acute Glaucoma (AG)

In POAG (Primary Open-Angle Glaucoma), the IOP increases slowly. But in acute angle-closure, it increases suddenly. This sudden rise in pressure can occur within a matter of hours and become very painful and can be associated with nausea and vomiting. Due to sudden IOP rise, there is no time for eye to adjust, hence, the eye becomes red, the cornea swells and cloudy, and see haloes around lights and develop blurred vision.

If untreated immediately, eyesight can be permanently destroyed with scarring of the trabecular meshwork and Cataracts development with optic nerve damage resulting in impaired vision. Many of these sudden attacks occur in darkened rooms, such as movie theaters due to papillary dilatation further narrowing angle and trigger an attack. Pupil dilates when a person is excited or anxious or stressed out due to fight or flight hormone release with initiation of this type of glaucoma attacks.

Many drugs such as anti-depressants, cold medications, antihistamines, and some medications to treat nausea can also cause dilation of the pupil and can lead to an AG attack. An acute attack may be stopped with a combination of drops which constrict the pupil, and drugs that help reduce the eye's fluid production. As soon as the IOP has dropped to a safe level, your ophthalmologist will perform a laser iridotomy so as to make a small opening in the iris to allow aqueous humor to flow without any impediment. These patients are treated with medication, such as pilocarpine, to avert an acute attack.

Pigmentary Glaucoma (PG)

Pigmentary glaucoma is an inherited open-angle glaucoma seen in near sighted men between the ages of 2-30. Myopic (nearsighted) eyes have a concave-shaped iris which creates an unusually wide angle. This causes the pigment layer of the eye to rub on the lens. This rubbing action causes the iris pigment to shed into the aqueous humor and onto neighboring structures, such as the trabecular meshwork which plug the pores of the trabecular meshwork, blocking aqueous humor drainage; thus increasing the IOP. Miotic therapy is the treatment of choice and the Laser iridotomy is being investigated.

Exfoliation Syndrome (ES)

This type of glaucoma is found all over the world, but common among people of European descent. A whitish material, which looks like tiny flakes of dandruff, builds up on the lens of the eye. This exfoliation material is rubbed off the lens by movement of the iris and at the same time, pigment is rubbed off the iris also. Both pigment and exfoliation material clogs the trabecular meshwork, leading to both open-angle glaucoma and angle-closure glaucoma. The person who has exfoliation syndrome, has about six times the chance of developing glaucoma in one eye.

Childhood Glaucoma (CG)

Childhood glaucoma is an infrequent abnormal eye disease; an important cause of childhood blindness; and may be associated with other medical disorder. Primary congenital glaucoma results from abnormal aqueous humor drainage system in about 1 out of 10,000 births in the United States. It is the most common form of glaucoma in infants. Ten percent of primary congenital glaucoma's are present at birth, and 80 percent are diagnosed during the first year of life. The pediatrician or family first notice eye signs of glaucoma including clouding and/or enlargement of the cornea. The elevated IOP can cause the eyeball itself to enlarge and injury to the cornea, poor vision, light sensitivity, tearing, and blinking. Most children need surgery (goniotomy, trabeculectomy and glaucoma drainage tubes) early to reduce IOP to increase the outflow of fluid from the eye; or decrease the production of fluid from the ciliary body. Up to 15 percent CG result in blindness in spite of aggressive anti glaucoma therapy.

Herpes Zoster of the Eye and Use of our Invention to Treat this Painful Condition

In the United States About 50,000 Herpes Zoster Ophthalmicus are reported every year resulting in herpes simplex epithelial keratitis. Herpes zoster ophthalmicus can be painful and result in complications centered on the destruction of the ocular structures that can lead to glaucoma and permanent loss of sight. Frequently affected structures are: Eyelids, conjunctiva, episclera, and sclera: Periorbital and conjunctival edema, focal scleral atrophy (late); secondary Staphylococcus aureus infection (1-2 wk); scarring that leads to incomplete eyelid closure leading to corneal exposure and desiccation (latent); with keratitis of various kinds leading to stromal keratitis; neurotrophic keratopathy (erosions, persistent defects, corneal ulcers) and Uveitis leading to glaucoma and cataract.

Antiviral agents used treat are acyclovir, valacyclovir, and famciclovir taken orally. These agents interfere with DNA synthesis and inhibit viral replication. Topical steroids may exacerbate spontaneous recurrences and occasionally, steroids may be prescribed to reduce inflammation. Topical trifluridine (Viroptic), which inhibits DNA synthesis, is used as eye drop 2-3 times a day. Eye drops, such as atropine are used to keep the pupil dilated, to help prevent a severe form of glaucoma, and to relieve pain. Even today there is no effective therapy for the local effects of this condition. The antiviral eye drops with insulin will be very effective in blocking the virus within the cell from multiplying and ameliorate the condition fast, reduce the possibility of developing glaucoma and relive the pain at the same time.

Treatment Modalities Presently Available that can be Combined with Our Invention to Enhance their Effectiveness

When a patient is diagnosed with glaucoma, the specialist physician will recommend one or a combination of the following treatment options: 1. Local and systemic therapeutic agents; 2. Laser Therapy 3. Surgery depending upon the stage of the disease its response to various therapeutic interventions. The treatment for Chronic Narrow-Angle, Chronic Open-Angle Glaucoma, and Open-Angle Glaucoma in the aged who have cataract; is to remove the enlarged lens and replace with an artificial lens which may widen the anterior chamber, relive pressure on the iris and relieve the condition.

Currently there are five major classes of medications that are used to lower the IOP in glaucoma: 1. β-adrenergic antagonists, 2. adrenergic agonists, 3. parasympathomimetics, 4. prostaglandin-like analogues and 5. Carbonic anhydrase inhibitors (Medeiros, et al., “Medical Backgrounders: Glaucoma”, Drugs of Today 2002; 38:563-570). The main drugs used to treat glaucoma are described by Peter R. Lewis, M.D. T. Grant Phillips, M.D. Joseph W. Sassani, M.D. Titled under the “Topical Therapies for Glaucoma: What Family Physicians Need to Know in the Journal of AAFP; Apr. 1, 1999” publication and others are herein incorporated (Rosenberg L F. Glaucoma: early detection and therapy for prevention of vision loss. Am Fam Physician 1995; 52:2289-98. Quigley H A. Open-angle glaucoma. N Engl J Med 1993; 328:1097-106. Primary open-angle glaucoma. San Francisco: American Academy of Ophthalmology, 1992:1-38. Liesegang T J. Glaucoma: changing concepts and future directions. Mayo Clin Proc 1996; 71:689-94. The management of primary open-angle glaucoma. Drug Ther Bull 1997; 35:4-6. Higginbotham E J. Initial treatment for open-angle glaucoma—medical, laser or surgical? Medication is the treatment of choice for chronic open-angle glaucoma. Arch Ophthalmol 1998; 116:239-40).

These publications describe the FDA approved diverse therapeutic agents to treat glaucoma which are incorporated herein along with our invention. When glaucoma is diagnosed, whether in the presence of normal or elevated IOP, it is treated with local eye drops as first-line therapy. The agents currently used to treat glaucoma are designed to decrease IOP by: 1. reducing the aqueous humor production in the ciliary body and processes; 2. to enhance aqueous outflow through the trabecular meshwork and at uveoscleral pathways.

First, a single topical therapeutic agent is prescribed starting with its smallest dose, then increasing to its maximum dosage till the desired effects are obtained, before another agent is added or a different agent or methods are selected to lower the unresponsive IOP. Numerous combinations of eye drops have been marketed and/or are undergoing trials that will simplify dosage regimens and thereby increase patient compliance. Our invention will reduce the dosage of the selected anti glaucoma agent, making them less likely to cause local or systemic complications.

Commonly used medications to lower the IOP are diverse and they can be combined with our invention to enhance the effectiveness. Therapeutic agents administered to the eye can pass rapidly through the nasolacrimal duct into the nose and then through the highly vascular nasal mucosa to enter the systemic circulation bypassing the liver; may result in adverse effects, even death in rare instances. We advise in this invention simple method to prevent the passage of the therapeutic agents passing to the nose (FIG. 6).

The broad class of therapeutic agents used in the treatment of glaucoma are summarized as follows:

1. Drugs to increase uveoscleral outflow of aqueous humor: Prostaglandin analogs like latanoprost (Xalatan), bimatoprost (Lumigan) and travoprost (Travatan) and Rescula. Bimatoprost in addition also increases trabecular meshwork outflow

2. Pharmacological therapeutic agents to decrease aqueous humor production by the ciliary body and ciliary process: Topical beta-adrenergic receptor antagonists such as: Timoptic, Timoptic (XE/GFS, or Ocudose), Betoptic, Optipranolol, Ocupress) timolol, levobunolol (Betagan), and betaxolol. They are either non selective like Timopitc or selective blockers such as Betopitc.

3. Therapeutic agents which have dual mechanism of decreasing aqueous production and increasing trabecular outflow: Alpha2-adrenergic agonists such as brimonidine (Alphagan, Iopidine, Propine).

4. Drugs which increase the outflow of aqueous humor through trabecular meshwork and possibly through uveoscleral outflow pathway, probably by a beta2-agonist action: Less-selective sympathomimetics like epinephrine and dipivefrin (Propine)

5. Medications which cause Contraction of the ciliary muscle (miotic), thus tightening the trabecular meshwork and allowing increased outflow of the aqueous humour. Miotic agents (parasympathomimetics) like pilocarpine and Ecothiopate used in chronic glaucoma.

6. Therapeutic agents that lower secretion of aqueous humor by inhibiting carbonic anhydrase in the ciliary body and process: Carbonic anhydrase inhibitors like dorzolamide (Trusopt), brinzolamide (Azopt), acetazolamide (Diamox). Oral carbonic anhydrase inhibitors are also available (Diamox, Neptazane).

7. Ophthalmic preparations that cause constriction of pupils to widen the iridocorneal angle: Physostigmine is used to treat glaucoma (and delayed gastric emptying). Instillation of 0.25% physostigmine sulphate eye drops caused a sustained miosis, with some deleterious action on the visual system.

8. Combination anti glaucoma therapeutic agent: Currently combinations of Timoptic and Trusopt (Cosopt) are available and are effective in twice a day dosing. Combinations of Xalatan and Timoptic as well as Alphagan and Timoptic are being studied and will be marketed soon. Combination eye drops may improve compliance if more than one drug is needed to control the IOP as it is more convenient to deal with just one bottle.

9. Combining our invention with the above described therapeutic agents will be more efficacious in controlling the glaucoma especially when it is resistant to single therapeutic agents and adverse systemic effects are anticipated.

The following table (Table 1) summarizes the popular glaucoma medications and their site of action

TABLE 1

| | Reduce aqueous | Increase Trabecular | Increase Uveoscleral |
|---|---|---|---|
| | humor production | outflow | outflow |
| 1 | Timoptic | Lumagan | Xalatan |
| 2 | Truspot | Pilocarpine | Alphagan |
| 3 | Alphgan | | |

Combination eye drops: Alphagan and Timoptic; and Xalatan and Timoptic

Besides the FDA approved drugs, there are host of Natural compounds that can lower the IOP in glaucoma. They are oral intake of: fish oil and omega 3 fatty acids, bilberries, vitamin E, cannabinoids (marijuana), carnitine, coenzyme Q10, curcurmin, Salvia miltiorrhiza, dark chocolate, erythropoietin, folic acid, Ginkgo biloba, Ginseng, L-glutathione, grape seed extract, green tea, magnesium, melatonin, methylcobalamin, N-acetyl-L cysteine, pycnogenols, resveratrol, quercetin and salt, (Ritch R (June 2007). “Natural compounds: evidence for a protective role in eye disease”. Can J Ophthalmol. 42 (3): 425-38. doi:10.3129/107-044. PMID 17508040. Tsai J C, Song B J, Wu L, Forbes M (September 2007). “Erythropoietin: a candidate neuroprotective agent in the treatment of glaucoma”. J Glaucoma 16 (6): 567-71. doi:10.1097/IJG.0b013e318156a556. PMID 17873720. Mozaffarieh M, Flammer J (November 2007). “Is there more to glaucoma treatment than lowering IOP?” Sury Ophthalmol 52 (Suppl 2): S174-9). Magnesium, ginkgo salt and fludrocortisone, are also used by some physicians.

Marijuana and IOP: Studies in the 1970s on Marijuana smoking and its effect on the IOP showed that marijuana (Cannabis) smoking lowered intraocular pressure (American Academy of Ophthalmology. Complementary Therapy Assessment: Marijuana in the Treatment of Glaucoma. Retrieved Sep. 30, 2008). In an effort to determine whether marijuana, or drugs derived from marijuana, might be effective as a glaucoma treatment, the US National Eye Institute supported research studies from 1978 to 1984. These studies demonstrated that some derivatives of marijuana lowered intraocular pressure when administered orally, intravenously, or by smoking, but not when topically applied to the eye.

In 2003 the American Academy of Ophthalmology released a position statement which said that “studies demonstrated that some derivatives of marijuana did result in lowering of IOP when administered orally, intravenously, or by smoking, but not when topically applied to the eye. The duration of the pressure-lowering effect is reported to be in the range of 3 to 4 hours.” This study indicates that anti glaucoma effect are due to CNS action and it can be due to active ingredients carried to aqueous humor production and histological structure of the eye through the blood to the eye after smoking.

Effect of Exercise on intra ocular Pressure: Physical exercise can reduce the IOP by 2 mm Hg and may not be effective in everyone. Isokinetic exercises cause considerable reduction in intraocular pressure and may be helpful in glaucoma (Ophthalmologica 1999; 213: 290-4). Aerobic exercise and a brisk walk also lower intraocular pressure (IOP) and short-term studies show it may improve blood flow to the retina and optic nerve as well.

Can acupuncture be used to treat IOP?: Two separate series of studies with glaucoma have found that most patients report a subjective improvement of central visual acuity after acupuncture but there was no change in IOP or visual field (Survey of Ophthalmology 2001:46:43-55). In my experimentation, I do believe that it is of no use in reducing the IOP in glaucoma or at best its effects are temporary in our experience.

Glaucoma and its relation to drinking Coffee (caffeine): Most physicians' advice glaucoma patients to avoid caffeine beverages. A recent study investigated the effect of caffeine on eye pressure (Ann Pharmacother. 2002; 36:992-5). The consumption of regular (180 mg caffeine in 200 mL beverage) and decaffeinated coffee (3.6 mg caffeine in 200 mL beverage) was compared. It is important to note that the eye pressure rise will depend on how much caffeine you consume. Regular coffee consumption of a 180 mg caffeine beverage resulted in about 3 mm Hg eye pressure increase in 1 hour (Ophthalmology. 1989; 96:624-6). Beverages containing about 50 mg caffeine may be fine. Tea (black or green—made of leaves) contains less caffeine and has the additional advantage of being rich in flavonoids (antioxidants good for the heart and the eye too), therefore tea may be a good alternative beverage for glaucoma patients.

Approach to Pregnancy and Glaucoma treatment: All existing glaucoma medications cross the placenta and are secreted into the milk except the one described in our invention below. The safest option is to avoid all glaucoma eye drops and seek alternate methods of reducing the IOP such as laser or incisional surgery. If eye drops are used, then select the drug doses with the lowest systemic levels in the mother. Timolol and Trusopt are approved by the American Academy of Pediatrics for use during lactation and be used with punctual occlusion of the lacrimal ducts (FIG. 5, 6) at the nose. Alphagan should not be used in infants and is best avoided during pregnancy and lactation. Xalatan has the potential of causing premature labor, therefore may not be a first choice agent (Stamper. Sury Ophthalmol 2002; 47:63-67, Johnson. Sury Ophthalmol 2001; 45:449-54).

The Description of some of the Therapeutic Agents Used to Treat Glaucoma which can be Combined with Our Invention for Effective Treatment of Glaucoma

The following anti glaucoma therapeutic agents' descriptions are incorporated here in from “The Eye Digest”, published on the internet on Apr. 15, 2010. These local anti glaucoma therapeutic agents are incorporated in our invention to be used with insulin to enhance (augmentation—amplification effects) the effectiveness and reduce the dose to prevent any local and systemic effects.

BETA BLOCKERS: Beta blockers lower IOP by decreasing aqueous humor production in the ciliary body-ciliary process and a slight increase in aqueous outflow. There are many useful beta-blockers e.g. betaxolol, timolol, befunolol, labetalol, propranolol, bupranolol, metaprolol, bunalol, esmalol, pindolol, carteolol, hepunolol metipranolol, celiprolol, azotinolol, diacetolol, acebutolol, atenolol, isoxaprolol which can be used in glaucoma in the treatment. Topically; beta blockers have been used as glaucoma therapy for two decades. Timolol maleate (top) is the standard agent against which other medications are measured in terms of efficacy, side effects and cost (Sorensen S J, Abel S R. Comparison of the ocular beta-blockers. Ann Pharmac 1996; 30:43-54).

Timolol and other topically applied beta blockers have been associated with asthma exacerbation, including status asthmaticus, worsening congestive heart failure, heart block and, rarely, sudden death and may block the typical systemic manifestations of hypoglycemia (Nelson W L, Fraunfelder F T, Sills J M, Arrowsmith J B, Kuritsky J N. Adverse respiratory and cardiovascular events attributed to timolol ophthalmic solution, 1978-1985. Am J Ophthalmol 1986; 102:606-11). Betaxolol (Betoptic), a cardio selective beta blocker, has a more favorable cardiopulmonary side effect profile than timolol and provides superior visual field preservation. Calcium channel antagonistic effect of Betoptic is said to provide neuroprotection. Despite lesser lowering of eye pressure than Timoptic, Betoptic has been reported to have a better effect on preservation of visual fields than Timoptic. Other topically applied beta blockers include metipranolol (Optipranolol), carteolol (Ocupress) and levobunolol (Betagan). The manufacturer of carteolol claims that the drug has an intrinsic sympathomimetic effect; therefore, it theoretically may have fewer cardiopulmonary side effects than timolol. Gel-forming solution of timolol maleate (Timoptic-XE) has the advantage of once-daily dosing. A patient with primary open-angle glaucoma and wheezing may be having a drug reaction to a topical beta blocker rather than new-onset asthma.

MIOTICS: The Miotics promote increased trabecular aqueous outflow by contracting the ciliary muscle of the eye (constricts pupils in addition). Pilocarpine (Isopto, Carpine), is inexpensive acetylcholine agonists and cholinesterase inhibitors miotic. It was isolated from the leaves of Pilocarpus plants in the 19th century. It has many side effects such as accommodative spasm; brow-ache and myopia are more pronounced in younger patients. In patients with cataracts, miotics may contribute to functional disability by decreasing daytime and, perhaps more significantly, nighttime vision. Systemic cholinergic effects such as nausea, vomiting, sweating and cutaneous vasodilatation may occur. Pilocarpine in a continuous-release vehicle (Ocusert Pilo) applied once weekly to the lower conjunctival sac can be used but it can fall out of the eye.

CARBONIC ANHYDRASE INHIBITORS: They act by lowering aqueous humor production. Oral carbonic anhydrase inhibitors are used in the management of primary open-angle glaucoma refractory to other forms of medical therapy for decades. Agents such as acetazolamide (Diamox) and methazolamide (Neptazane) decrease aqueous humor secretion by the ciliary epithelium. It causes general malaise, symptomatic metabolic acidosis, renal calculi and bone marrow suppression, significant hypokalemia. Concomitant use with aspirin increases the risk of salicylate toxicity. Dorzolamide (Trusopt) and brinzolamide (Azopt) are the first topical carbonic anhydrase inhibitors labeled by the U.S. Food and Drug Administration (FDA) for the treatment of primary open-angle glaucoma applied two to three times daily. Dorzolamide is also marketed in combination with timolol (Cosopt) for local eye drops with least systemic side effects. Like acetazolamide, dorzolamide and brinzolamide are sulfonamide derivatives, hence bone marrow dyscrasias, transaminitis (elevated transamases) and dermatologic reactions besides bitter taste (experienced by up to 25 percent of patients), headache, nausea, asthenia and fatigue and may even nephrolithiasis may occur.

SYMPATHOMIMETIC THERAPEUTIC AGENTS: Topical sympathomimetics decrease aqueous production or increase aqueous outflow. They may be divided into epinephrine (alpha- and beta-receptor stimulation) and clonidine-like agents—a pure alpha-2-receptor agonist which has 183 times more affinity for alpha 2 than alpha 1 receptors. Dipivefrin (Propine), an epinephrine prodrug, is taken twice daily. Although dipivefrin produces fewer ocular and systemic side effects than epinephrine, it is being supplanted by clonidine-like agents for glaucoma therapy. The FDA has labeled apraclonidine (Iopidine) for use in the management of transient IOP elevations after ocular surgery. Brimonidine (Alphagan) is approved for maintenance glaucoma treatment and may be suitable as monotherapy with fewer CNS and ocular side effects than apraclonidine. The increased selectivity of brimonidine for alpha₂-receptor sites is postulated to decrease IOP by limiting aqueous production and facilitating increased outflow via the uveoscleral pathway.

PROSTAGLANDIN ANALOGS: Prostaglandins are metabolite product of arachidonic acid in the body. Arachidonic acid is converted to prostaglandin G_{2,} which is consequently converted to prostaglandin H₂. A number of different types of prostaglandins are known in the art including A, B, C, D, E, F, O, I and I-Series prostaglandins (EPO 561 073A1). There are many inventions of prostaglandin derivatives and analogs which will lower IOP without undue side effects. For example, U.S. Pat. Nos. 5,151,444; 5,422,368; 5,688,819; and 5,889,052 describe prostaglandin derivatives and analogs said to exhibit reduced side effects and enhanced therapeutic profiles. The contents of the foregoing patents are by this reference incorporated herein.

The developments of a prostaglandin suitable for clinical use in the treatment of glaucoma were previously hindered by the occurrence of ocular side effects, primarily conjunctival hyperemia. Latanoprost (Xalatan-F₂ₐₗₚₕ) was recently approved for use in patients with glaucoma, taken once daily at bedtime with least local and systemic side effect profile. Latanoprost lowers IOP by increasing uveoscleral outflow -the minor pathway for the removal of aqueous humor from the anterior chamber of the eye with a sustained IOP-lowering effect throughout the day and night. Increased iris pigmentation occurs in up to one in 17% of the patients treated with latanoprost. The color change is stable and may not be reversible with discontinuation of the drug. Lash growth, another documented ocular side effect of latanoprost, is a cosmetic significance. Although the newer topical anti-glaucoma agents (brinzolamide, brimonidine, dorzolamide, 15-Keto-latnaoprost and latanoprost) are effective in reducing intraocular pressure, further research is needed to determine their effect in visual field preservation, which is the treatment goal for glaucoma in our invention.

These results suggest that 15-keto acid of latanoprost, a 13,14-dihydro-15-keto type metabolite, produced from latanoprost in the eye after instillation of latanoprost participates in the maintenance of the intraocular pressure lowering effect after instillation of latanoprost (U.S. Pat. No. 6,596,765 B2). U.S. PATENT APPLICATION PUB. NO.: 2002/0193441 A1 discloses the methods for the treatment of glaucoma and/or ocular hypertension in humans utilizing improved doses of certain prostaglandin derivatives and analogs are disclosed. The method of claim wherein the prostaglandin is selected from the group consisting of latanoprost, travoprost, unoprostone isopropyl, and bimatoprost.

U.S. Pat. No. 6,458,836 B1 discloses treatment of ocular hypertension and glaucoma by long-term therapy with a prostaglandin related compound for eliminating or reducing potential iridic pigmentation. Composition useful for the treatment and use of the prostaglandin related compound for producing the composition are also disclosed. Now, it has been found that a prostaglandin related compound of the present invention which substantially does not stimulate the prostaglandin FP receptor or possesses an FP specific affinity one-tenth or less than that of latanoprost and is otherwise usable as a topically administered ocular hypotensive, can be safely administered to humans over prolonged time periods without causing dark colored iridic pigmentation. Certain of these compounds are those in which carbon atom number 15 is substituted by an oxo group (15-keto compounds), or those in which carbon atom number 15 is substituted by a hydroxy group and the omega chain beyond carbon atom number 15 contains a straight chain of at least 6 carbon atoms or a straight chain of at least 3 carbon atoms with a ring at the terminal of the omega chain. As above discussed, the compounds of this invention can safely be administered topically for ocular hypotensive effect to human patients over prolonged time periods without causing the brown iridic pigmentation.

Future directions anti glaucoma therapeutic agents: Nitrates and calcium channel blockers are undergoing evaluation for the management of primary open-angle glaucoma (Wilson M R, Gaasterland D. Translating research into practice: controlled clinical trials and their influence on glaucoma management. J Glaucoma 1996; 5:139-46). Interest in these vasodilators stems from the hypothesis that impaired ocular blood flow contributes to the optic nerve damage that leads to glaucoma. Our invention can play a major role in correcting the deficient blood flow. Investigator are studying the role of trabecular meshwork protein (identified as the TIGR protein) which may play in the pathogenesis of primary open-angle glaucoma (Stone E M, Fingert J H, Alward W L, Nguyen T D, Polansky J R, Sunden S L, et al. Identification of a gene that causes primary open angle glaucoma. Science 1997; 275: 668-70).

Although most medications are applied topically to the eye, they can cause severe systemic side effects and adversely affect the quality of the patient's life. Examples are: Miotics which constrict the pupils may reduce the patient's visual acuity, particularly in the presence of lenticular opacities. Topical beta blockers such as Timolol™ are associated with systemic side effects such as fatigue, confusion, asthma, and exacerbation of cardiac symptoms especially if they are withdrawn rapidly. Oral administration of acetazolamide; a carbonic anhydrase inhibitors, can be associated with systemic side effects including chronic metabolic acidosis.

The drug therapies for glaucoma are sometimes associated with significant side effects, such as headache, blurred vision, allergic re actions, potential interactions with other drugs and death from cardiopulmonary complications. Our invention reduces the dose of these eye drops and reduces or eliminates the serious adverse effects. Our invention reduces the possibility of these adverse effects by reducing the doses of these therapeutic agents. Application of pressure at the lacrimal out flow ducts as shown in FIG. 6 further helps to reduce the nasal mucosal contact and systemic effects.

When the medical treatment fails and vision is threatened by elevated IOP, the alternative therapy are laser treatment (laser trabeculoplasty), and surgical therapy (trabeculectomy or trabeculotomy), or the like is performed.

Selective Laser Trabeculoplasty (SLT): There are many types Laser surgery performed such as: Laser trabeculoplasty, Nd:YAG laser peripheral iridotomy (LPI), Diode laser cycloablation. And for Glaucomatous painful Blind Eye and some cases of Glaucoma, Cyclocryotherapy to ablate the ciliary body could be considered. SLT (Selective Laser Trabeculoplasty) is a safe and simple in-office laser treatment that effectively reduces the pressure in the eye for most patients with glaucoma. It uses an advanced laser system to target only those containing melanin, a natural pigment. This allows for only these cells to be affected, leaving surrounding tissue intact. As a result, your body's own healing response helps lower the pressure in your eye.

Argon Laser Trabeculoplasty (ALT): ALT is also an in-office laser therapy similar to that of SLT, but uses longer pulses that non-selectively target cells; more likely to result in unnecessary damage and scarring to surrounding internal eye structures, limiting treatment repeatability options for maintaining eye pressure control and vision preservation. If non-surgical methods fail to lower intraocular pressure (IOP) to a safe level, surgery should be performed. The two major types of surgical interventions are:

Trabeculectomy (most common glaucoma surgical intervention): If treatment with laser therapy or medications does not lower intraocular pressure (IOP) to a safe level, surgery is the next choice by to make a new drain in the eye called a trabeculectomy. The surgeon removes a tiny section of the wall of the eye which may include the trabecular meshwork. This procedure opens a newly created drain which creates allows for direct bypass of fluid instead of relying solely on the natural mechanism of the trabecular meshwork to reduce eye pressure. Fluid can now drain much more efficiently through the new opening into a reservoir (bleb) underneath the conjunctiva. The fluid is then absorbed by the body. If successful, lower eye pressure will be attained and hopefully the eye will not have any further glaucoma damage and vision will be maintained. This surgery results in approximately 70% success of operated eyes and will have satisfactory eye pressure and no need for medication one year after surgery. If medications are added, over 90% of treated eyes will have a satisfactory lowering of eye pressure.

Non-penetrating Filtration Surgery (novel surgical intervention); also referred to as a Deep Sclerotomy which creates a thin area on the outer layer of the eye to reduce eye pressure. The non-penetrating deep sclerectomy can be an effective alternative to traditional trabeculectomy. This procedure is basically a trabeculectomy with the final thin layer left intact where the aqueous humor can be absorbed directly into the blood vessels to the space under the top of the conjunctival layer (FIGS. 3, 5A) to be absorbed like traditional surgery. This procedure reduces the risk of creating too low of pressure in the eye (hypotony) with lowered chances for infection. Because of the learning curve associated with nonpenetrating surgery, the surgeon's experience can significantly affect his or her success rate.

Nearly 100,000 trabeculectomies are performed on Medicare-age patients per year in the United States which is associated with failure (up to 15%), infection (2-5%) choroidal hemorrhage, visual loss (1%), cataract formation, hypotomy maculopathy resulting in visual loss due low IOP. All surgical procedures have risks; such as infection, bleeding, swelling in the retina, development of fluid under the retina (choroidal detachment), retinal detachments etc.

There are several glaucoma drainage surgical implants developed to drain the aqueous humor. These include the original Molteno, the Baerveldt tube shunt, or the valved implants (Ahmed glaucoma valve implant or the ExPress Mini Shunt, pressure ridge Molteno implants). The scarring over the conjunctival dissipation segment of the shunt may become too thick for the aqueous humor to filter through. This may require preventive measures using anti-fibrotic medication like 5-fluorouracil (5-FU) or mitomycin-C (during the procedure), or additional surgery. Our invention of using insulin with anti fibrotic chemotherapeutic medications may completely eliminate and/or drastically reduce the scarring and keep open the shunt for long time.

In spite of all the treatment modalities evolved over decades as described above, there is still no a cure for glaucoma and elevated IOP. Many therapeutic agents have been proposed to treat glaucoma and many are on the horizon. Our invention independently and/or with known therapeutic, pharmaceutical, biochemical and biological agents or compounds will further help in curtailing or curing the glaucoma and raised IOP.

There are problems with the aforementioned approaches to treating glaucoma in that the treatments can be accompanied by side-effects. For example, the instillation of a cholinergic agent, such as pilocarpine, into the eye of a subject can cause nausea, diarrhea, muscular spasms, sweating, lacrimation, salivation, etc. Contraction of the pupil (miosis) and of the ciliary muscle of the eye, as well as dilation of the blood vessels of the iris and conjunctiva also can be observed. Visual complications, e.g., spasm of accommodation, myopia or a decrease in visual acuity, also can occur. The treatment with a sympathomimetics agent such as dipivalylepinephrine is known frequently to produce sensations of burning or irritation in a subject. Another side-effect of these agents is the appearance of cardiac disturbances, e.g., palpitations, tachycardia, arrhythmia, etc.

Glutamate induced excitotoxicity on the retinal ganglion cell in the retina in glaucoma patients' eyes: It is known that that the elevated glutamate levels are associated with glaucoma, which damage to retinal ganglion cells. This can be controlled by administering to the patient a compound capable of reducing glutamate induced excitotoxicity in a concentration effective to cause reduction of such excitotoxicity. Such a compound that is an antagonist of NMDA receptor-mediated excitotoxicity, in a concentration effective to cause reduction of said excitotoxicity, said antagonist being capable of crossing the blood brain barrier and the blood retina barrier are described. They list dozens of such compounds for use. Commercially available NMDA-receptor antagonists include ketamine, dextromethorphan, memantine, eliprodil, and amantadine. The opioids, methadone, dextropropoxyphene, and ketobemidone are also antagonists at the NMDA receptor.

U.S. Pat. No. 4,985,417 discloses treatment of glaucoma in a mammal wherein an effective amount of an active water soluble carbonic anhydrase inhibitor is administered.

U.S. PATENT APPLICATION PUB. NO.: US 2005/0014808 A1 discloses a pharmaceutical composition for the prophylaxis or treatment of glaucoma consisting of an angiotensin II antagonist (ACE inhibitors and angiotensin converting enzyme receptor blockers-ARBS) and dorzolamide or a pharmacologically acceptable salt thereof.

U.S. PATENT APPLICATION PUB. NO.: 2004/0176455 A1 discloses methods and kits for treatment of glaucoma. The methods of the invention include administering a therapeutically effective amount of a deprenyl compound to a subject such that the subject is treated for glaucoma.

U.S. PATENT APPLICATION PUB. NO.: 200910062400 A1 Disclosed the etiology of retinal damage; a method of reducing glaucoma and prevent retinal ganglion cell (RGC) death in a subject by administering to the subject an amount of R(+)-N-propargyl-1-aminoindan or a pharmaceutically acceptable salt thereof effective to reduce glaucoma. Glaucomatous optic neuropathy appears to result from specific pathophysiological changes and subsequent death of RGCs and their axons. The manner of RGC death is thought to be biphasic: a primary injury responsible for initiation of damage followed by a slower, secondary degeneration attributable to the hostile environment surrounding the degenerating cells (Kipnis, et al., “T Cell Immunity To Copolymer 1 Confers Neuroprotection On The Damaged Optic Nerve: Possible Therapy For Optic Neuropathies”, Proc Natl Acad Sci 2000; 97:7446-7451).

Although the activating the apoptosis of RGC has not been recognized. The deprivation of neurotophic factors, ischemia, chronic elevation of glutamate and disorganized nitric oxide metabolism are alleged to be possible mechanisms (Farkas, et al., “Apoptosis, Neuroprotection and Retinal Ganglion Cell Death: An Overview”, Int Opthalmol Clin 2001; 41:111-130). In addition, it is possible that the mechanisms leading to RGC death share common features with other types of neuronal injury, such as signaling by reactive oxygen species, depolarization of mitochondria, or induction of transcriptionally regulated cell death (Weinreb, et al., “Is Neuroprotection a Viable Therapy for Glaucoma?” Arch Opthalmol 1999; 117:1540-1544).

The anti-inflammatory drugs acetylsalicylate and rednisolone are known to regulate the microglia responsible for the onset of photoreceptor apoptosis and retinal degeneration thereafter; both drugs are proved to be ineffective. (Sam et al., Effect of steroidal and non-steroidal drugs on the microglia activation pattern and the course of degeneration in the retinal degeneration slow mouse, Ophthalmic Res. (2005) 37(2):72-82). Rasagiline, R (+bN-propargyl-1-aminohclan, is a potent second generation monoamine oxidase (MAO) B inhibitor may be effective in preventing the RGC death.

U.S. PATENT APPLICATION PUB. NO.: 200710092502 A1 inventions provides a method of treating glaucoma or preventing glaucoma in a person at risk of developing glaucoma, by applying to the eye of said person, an effective amount of an antibacterial agent having activity against the Helicobacter Pylori bacteria to thereby eradicate, inhibit and/or control said bacteria. It has been reported that levels of antibodies to Helicobacter Pylori found to be significantly higher in people with primary open-angle glaucoma and exfoliation glaucoma compared to cataract surgery patients in a prospective study. The authors of this study were reported to “believe that the bacteria may play a role in the pathobiology of these forms of glaucoma.

Since Helicobacter pylori has been implicated in causing gastritis and peptic ulcers and may be implicated in causing stomach cancer, various pharmaceutically-active compounds have been disclosed as useful for preventing and/or treating the conditions caused by the bacteria. These compounds are generally suitable for eradicating the causative bacteria in the gastrointestinal tract. The method uses antibacterial agent in combination of lactoperoxidase and a peroxide donor; rifabutin and a therapeutically effective amount of a second antibiotic or antimicrobial agent selected from the group consisting of amoxicillin, tetracycline and bismuth compounds; The method of claim 5 wherein said antibacterial agent further comprises a proton pump inhibitor.

U.S. Pat. No. 6,177,427 BI Compositions of non-steroidal glucocorticoid antagonists for treating glaucoma or ocular hypertension and methods for their use are disclosed. It is believed that the steroidal glucocorticoid antagonists bind to the glucocorticoid receptor in trabecular meshwork cells, and thereby prevent binding of endogenous glucocorticoid to the glucocorticoid receptor. They may also displace endogenous glucocorticoids bound to glucocorticoid receptors. Ketoconazole and clotrimazole are known glucocorticoid blockers. They are not known to be useful in treating or controlling glaucoma. They name numerous non-steroidal glucocorticoid antagonists with inactive ingredients with can be used in conjunction with our invention.

A variety of facilitators, carriers, adjuvant agents, absorption enhancers and facilitators, assist to get entry into the cell, potentiators of therapeutic action (augmentation—amplification effects), cell metabolic activity enhancers, cell multiplication enhancers and other methods have been used to enhance the absorption and/or to potentiate the effect of therapeutic, pharmaceutical, biochemical and biological agents or compounds administered to the patients for treatment of diseases. U.S. Pat. No. 2,145,869 by Dr. Donato Perez Garcia disclose a method for the treatment of syphilis in general and neurosyphilis in particular using subcutaneous insulin injections as enhancer of uptake of the arsenic therapeutic agents by the brain crossing the blood brain barrier. U.S. Pat. No. 4,196,196 discloses a composition of insulin, glucose and magnesium dipotassium ethylene diamine tetra acetic acid (EDTA) to enhance tissue perfusion and to facilitate a divalent/monovalent cation gradient uptake into the cells or out of cells.

Insulin in the intravenous infusion enhances the uptake and activity of potassium and magnesium at the cellular level. I have used this method in many surgical and post surgical patients to decrease the potassium level in the cells whenever there was low or high levels of potassium in the serum for 3 decades. The method is continued to be used in medical practice even today. U.S. Pat. No. 4,277,465 discloses an adjuvant to potentiate insulin for treatment of diabetes. U.S. Pat. No. 4,971,951 discloses Insulin Potentiation Therapy (IPT) for the treatment of virally related diseases such as herpes, AIDS and cancers using insulin to deliver the drugs inside the cell with less or non-toxic low doses of therapeutic agents, to enhance the uptake of therapeutic indicated and with increased non toxic doses. None of these describe the use of insulin and IGR-1 locally to treat glaucoma as described in this invention.

Besides Aspirin, antibiotics; insulin is the most commonly used therapeutic agent known to the public and professional people alike. It has been used in home by the patients or in the office by the physicians. It can be easily obtained by prescription and used for treating glaucoma as described in this invention. Insulin is a hormone secreted by beta cells in the islets of Langerhans in the pancreas. It activates and participates in all the metabolic pathways in the normal, disease afflicted cells; can lead to increased DNA, RNA and protein synthesis which result in increased growth by mitosis (Osborne C K, et al. Hormone responsive human breast cancer in long-term tissue culture: effect of insulin. Proc Natl Acad Sci USA. 1976; 73: 4536-4540); enhances the permeability of cell membranes to many therapeutic agents besides glucose, and electrolytes; it helps and facilitates to move the drugs and therapeutic agent molecules from extra cellular fluid (ECF) to intracellular fluid (ICE) meaning from outside the cells to inside the cells.

Insulin is anabolic trophic factor needed for the growth, multiplication, of all cells in the body including the healthy vascular endothelium, neurons in the retina and macula, as well as trabecular meshwork cells which are deficient in glaucoma as described above. Increased cellular metabolic activity induced by insulin also enhances the uptake and enhances the action of all therapeutic agents, biological and pharmacological agents by the cells and inside the cell including the cells responsible for healthy retina and production of glaucoma in the production and drainage of aqueous humor. Insulin enhances their concentration and effectiveness which has disease curing besides retinal function and trabecular meshwork. Once inside the cells; the insulin enhances, and augments the effectiveness of any and all therapeutic agents including the agent proven to treat glaucoma by inhibiting aqueous humor production and/or their drainage and the rest of the retina involved in glaucoma.

### SUMMARY OF THE INVENTION

A safe and effective treatment for glaucoma for mammalian species comprises the steps of applying insulin and/or insulin like growth factors (IGF-1) to an eye. In addition to the insulin, another therapeutic agent may be applied to enhance the activity of the insulin. The therapeutic agent may be a pharmaceutical agent or a biochemical pharmaceutical agent. The therapeutic agents include prostaglandin analogs, topical beta-adrenergic receptor antagonists-β blockers, Alpha2-adrenergic agonists hair growth therapeutic agents, beta2-agonist action agents, parasympathomimetic miotic agents, carbonic anhydrase inhibitors, and Physostigmine. In another embodiment, a combination of at least two agents are applied to the eye. To enhance the effect of the insulin, uptake facilitators may be used. Additionally, an antibacterial agent may be applied to control bacterial infection.

### BRIEF DESCRIPTION OF THE DRAWINGS

FIG. 1 is a diagram of an entire eyeball showing the structure therein.

FIG. 2 shows Histological section of the anterior ⅓ of the eye ball showing the structures involved in the production of aqueous humor from the ciliary body to and its drainage at iridoslceral angle (modified form Grays' anatomy).

FIG. 3 is the diagram illustrating the structures involved in the production, circulation and drainage of the aqueous humor.

FIG. 4 shows the whole mount of the corneal endothelium with sporadic inter cellar pores which allow the aqueous humor to permeate the corneal stroma.

FIG. 5 is the diagrammatic section of the eye showing conjunctival sac and the iridocorneal angle involved in the treatment of glaucoma and the absorption of therapeutic agents from conjunctival sac through the complex vascular anastomosis and permeation through the tissues they come in contact.

FIG. 5A is a digramatic presentation section of the anteriour part of the eye shown in FIG. 5.

FIG. 6 is the diagram showing the route of drainage of the lacrimal fluid and therapeutic agents from the conjunctival sac to the nasal.

FIG. 7 is the diagram of the optic nerve with its membranes and arachoid villi, SAS and their relation to choroid.

### DETAILED DESCRIPTION OF THE INVENTION

Before explanation and description of the disclosed embodiments of the present invention in detail, it is to be understood that the invention is not limited in its application to the details of the particular examples and arrangement shown since the invention is capable of other examples and embodiments. Also, the terminology used herein is for the purpose of description and not of limitation. As earlier enumerated above and recited below; this application has been filed in order to disclose: Insulin and Insulin like Growth factor (IGF-1) have been found to have high activity against glaucoma and they enhance the effectiveness (augmentation—amplification effects) of therapeutic, pharmaceutical, biochemical and biological agents or compounds used in the treatment of glaucoma and elevated IOP in smaller does than indicated; which in turn reduces or eliminates their systemic adverse effects.

In the following detailed description of the invention, reference is made to the drawings and microphotographs in which reference numerals refer to like elements, and which are intended to show by way of illustration specific embodiments in which the invention we describe using insulin, and IGF-1 with or without other known anti glaucoma therapeutic, pharmaceutical, biochemical and biological agents or compounds enumerated here may be prescribed and practiced. It is understood that other embodiments may be utilized and that structural changes may be made without departing from the scope and spirit of the invention described herein.

At present, the insulin is exclusively used to treat diabetes, and our discoveries and inventions describes its use topically (locally) in other disease conditions other than diabetes including glaucoma and eye diseases. Insulin and its biological effects on healthy and disease afflicted cells; its role in uptake and augmentation—amplification effects of therapeutic, pharmaceutical, biochemical and biological agents or compounds on these cells are described herein.

Insulin is a metabolic activity enhancer of all cells and therapeutic agents. Hence it can play an important role in treatment many diseases including glaucoma. A synergy between certain membrane and metabolic effects of insulin on cell molecular biology increases therapeutic efficacy of all anti glaucoma therapeutic, pharmaceutical, biochemical and biological agents or compounds and it does so with reduced doses of the drugs, enhancing their uptake with augmented greater than before therapeutic efficacy and increasing safety in lowering the IOP and restoring the retinal function.

It is known that the pharmaceutically acceptable oxidizing agent facilitates the delivery of the bioactive agent through the skin and mucous membranes such as oral cavity, nasal passages and conjunctiva. In general, the oxidizing agent can react with molecules present in the conjunctiva would adversely react with the bioactive agent. For example, reduced glutathione present in the mucus membranes of the eyes and skin can inactivate bioactive agents such as insulin by breaking chemical molecular bonds. Not wishing to be bound by theory, when delivering insulin through the skin and conjunctiva, reduced glutathione can inactivate insulin. Specifically, insulin has numerous disulfide bonds which are crucial for its protein conformation, biological activity, and subsequent therapeutic effects.

Reduced glutathione will inactivate insulin by reducing or breaking insulin's disulfide bonds. Once these disulfide bonds are broken; insulin becomes inactive due to lost protein conformation and biological activity. Thus, the administration of the oxidant by eye drops (as described by Shantha et al in Transmucosal Delivery of Therapeutic Agents and Methods of Use Thereof, U.S. Patent Application Pub. No. 2009/0347776 A1, the entire contents of which are herein incorporated by reference) herein prevents the inactivation of the bioactive agent such as insulin when applied to the skin and conjunctival sac of the eye. Specifically, applying an oxidant or a pharmaceutically oxidizing agent to conjunctival sac will lower or prevent the effects reduced proteins and reduced biological molecules have on the bioactive agents. In this manner, the inactivation of bioactive agents via reduction or cleavage of crucial molecular bonds will be avoided.

The selection and amount of the pharmaceutically acceptable oxidizing agent can vary depending upon the bioactive agent that is to be administered. In one aspect, the oxidizing agent includes, but is not limited to, iodine, povidone-iodine, and any source of iodine or combinations of oxidants, silver protein, active oxygen, potassium permanganate, hydrogen peroxide, sulfonamides, dimethyl sulfoxide or any combination thereof. These oxidizing agents may also act as absorption agents which help facilitate delivery of a therapeutic agent onto and into a skin. In one aspect, the oxidant is at least greater than 1% weight per volume, weight per weight, or mole percent. In another aspect, the mucosal membrane permeability enhancer may be at least greater than 1.5%. 2.0%. 20 2.5%, 3.0%, 3,570, 4.0%, or 4.5% weight per volume, weight per weight, or mole percent. In this aspect, the oxidant may range from 2% to 10%, 2% to 9.5%. 3% to 8%, 3% to 7%. or 4% to 6% weight per volume, weight per weight, or by mole percent.

Our preliminary studies have shown that the conjunctiva unlike normal skin may not act as a barrier for entry of insulin due to the paucity of the presence of reduced glutathione. It is likely that conjunctiva hardly contains any insulin blocking agent, besides; it does not have the multilayered stratum coneium as seen on the skin which blocks the entry of insulin in the skin. The insulin deposited in the conjunctival sac is rapidly absorbed and reaches the trabecular meshwork and the ciliary body without being inactivated to exert its therapeutic effect.

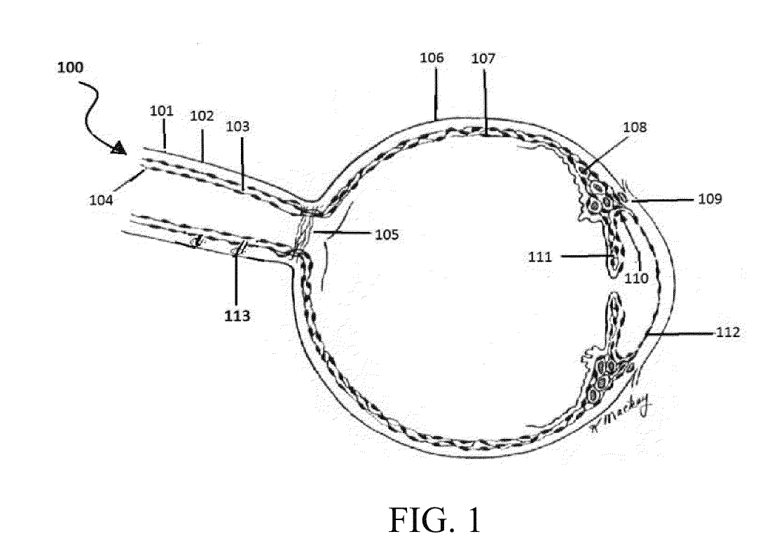

In one aspect, transconjunctival penetration of insulin and therapeutic, pharmaceutical, biochemical and biological agents or compounds can be facilitated by enhancers that can be used to further expedite the entry of these agents into the anterior chamber, trabecular meshwork, ciliary body, choroid and retina (FIGS. 1-5). Penetration enhancers not only penetrate a membrane efficiently, but these enhancers also enable other bioactive agents to cross a particular membrane more efficiently. Penetration enhancers produce their effect by various modalities such as disrupting the cellular layers of the conjunctival sac surface interacting with intracellular proteins and lipids, or improving partitioning of bioactive agents as they come into contact with the mucosal membranes.

With these enhancers, macromolecules up to 10 kDa are able to pass through the conjunctival sac layers of the eyes reaching the site of glaucoma where the blood vessels and retina are undergoing pathological changes. These enhancers should be non-toxic, pharmacologically inert, non-allergic substances. In general these enhancers may include anionic surfactants, ureas, fatty acids, fatty alcohols, terpenes, cationic surfactants, nonionic surfactants, zwitterionic surfactants, polyols, amides, lactam, acetone, alcohols, and sugars. In one aspect, the 10 penetration enhancer includes dialkyl sulfoxides such as dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penetration enhancer may include lauryl alcohol, diisopropyl sebacate, oleyl alcohol, diethyl sebacate. dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethylhexyl pelargonate, butyl benzoate, benzyl benzoate, benzyl salicylate, dibutyl phthalate, or any combination thereof. In one aspect, the skin permeability enhancer is at least greater than 1% weight per volume, weight per weight, or mole percent.

In another aspect, the mucosal membrane permeability enhancer may be at least greater than 1.5%, 2.0%, 2.5%, 3.0%. 3.5%. 4.0%. 4.5% up to 50% weight per volume, weight per weight, or mole percent. In one aspect, the mucosal membrane permeability enhancer is dimethyl sulfoxide. In this aspect, the amount of dimethyl sulfoxide may range from 2% to 10%. 2% to 9.5%. 3% to 8%. 3% to 7% or 4% to 6% weight per volume, weight per weight, by mole percent, or any effective therapeutic amount.

In other aspects, these additional components may include antiseptics, antibiotics, anti-vitals, anti-fungals, anti-inflammatories, anti-dolorosa, antihistamines, steroids, vasodilators and/or vasoconstrictors to reduce inflammation, irritation, or reduce rapid absorption through conjunctival sac. Such vasoconstrictors may include phenylephrine, ephedrine sulfate, epinephrine, naphazoline, neosynephrine, vasoxyl, oxyrnetazoline, or any 5 combination thereof. Such anti-inflammatories may include non-steroidal anti-inflammatory drugs (NSAIDs). NSAIDs alleviate pain and inflammation by counteracting Cyclooxygenase and preventing the synthesis of prostaglandins.

In one aspect. NSAIDs include celecoxib, meloxicam, nabumetone, piroxicam, napmxen, oxaprozin, rofecoxib, sulindac, ketoprofen, valdewxid, anti-tumor necrosis factors, 10 anti-cpokines, and anti-inflammatory pain causing bradykinins or any combination, thereof. Such antiseptics, anti-vitals, anti-fungals, and antibiotics, may include ethanol, propanol, isopropanol, or any combination thereof; a quaternary ammonium compounds including, but not limited to, benzalkonium chloride, cetyltrimethylammonium bromide, cetylpyridinium chloride, benzethonium chloride, or any combination thereof; boric acid; chlorhexidine gluconate, hydrogen peroxide, iodine, mercurochrome, ocetnidine dihydrochloride, sodium chloride, sodium hypochlorite, silver nitrate, colloidal silver, mupirocin. erthromycin, clindamycin, gentamicin, polymyxin, bacitracin, silver, sulfadiazine, or any combination thereof. It is intent of this invention to use of the insulin along with above described anti-inflammatory antibacterial agents that can eliminate of the causes of glaucoma and restore normal sight.

Treatment with local ophthalmic therapeutic agents that are used in the treatment of glaucoma at present time can be accompanied by local and systemic undesirable side-effects. For example the instillation of a cholinergic agent, such as pilocarpine, into the eye of a subject can cause nausea, diarrhea, muscular spasms, sweating, lacrimation, salivation, etc; Contraction of the pupil (myosis) and of the ciliary muscle of the eye, as well as dilation of the blood vessels of the iris and conjunctiva also can be observed. Visual complications, e.g., spasm of accommodation, myopia or a decrease in visual acuity.

The treatment with a sympathomimetics agent such as dipivalyl epinephrine is known frequently to produce sensations of burning or irritation in a subject as well as palpitations, tachycardia, arrhythmia, etc.; Clonidine, a known as an alpha-2-adrenergic receptor agonist, can bring about mydriasis; as well as an initial phase of ocular hypertension (biphasic effect). Furthermore, systemic effects, such as bradycardia and hypotension, have been observed. The use of beta-blocking medicaments also can cause important systemic effects after topical administration to the eye, due to the absence of a “first pass effect” and one should extreme precautions when used in those with cardiac or pulmonary functional disorders (arrhythmia, cardiac arrest, asthma, dyspnea and bronchospasms). Suicidal depression, hallucinations, nightmares or psychoses requiring hospitalization have been reported. Sympatholytic eye drops such as guanethidine, causes hyperemia of the conjunctiva and irritation, not to mention the fact that these agents only have a low tendency to reduce intraocular pressure. The treatment of glaucoma with carbonic anhydrase inhibitors, such as acetazolamide or methazolamide, can result in depression of the central nervous system, weight loss and, mainly, bone marrow depression.

The use of conventional hypotensive agents for the treatment of glaucoma is accompanied by considerable risks. Known medications are not particularly well suited for topical treatment and the systemic side-effects of these medicaments can have, in some cases, severe consequences. Using insulin as described in this invention obviates the use of full dose of the medications and helps to reduce the above described complication to the minimum or no complications. Our invention of use Insulin eye drops itself does not have any of the above effects; it does not change the blood glucose levels either, and it reduces the dose of the traditional anti glaucoma therapeutic agents which are already in use thus reducing the drug related adverse effects.

In accordance with one aspect of the invention, the compound used to apply locally to the eye lids site are mixed with a conjunctivally well-suited vehicle or carrier. The compositions of this invention may comprise aqueous solutions such as e.g., physiological saline, oil, gels, patches, solutions or ointments. The vehicle which carry these biologically active therapeutic agents may contain conjunctivally compatible preservatives such as e.g., benzalkonium chloride, surfactants like e.g., polysorbate 80, liposome's or polymers, for example, methyl cellulose, polyvinyl alcohol, polyvinyl pyrrolidone and hyaluronic acid etc. We do use sterile water or normal saline in our preparation.

There are various forms of insulin used to treat diabetes which can be formulated to be used in our invention. They are grouped under rapid, short, intermediate, and long acting insulin. It is also dispensed as premixed form containing rapid to long acting insulin. Insulin products are categorized according to their putative action profiles as:

1. Rapid-acting: insulin lispro, insulin aspart, and insulin glulisine

2. Short-acting: regular (soluble) insulin

3. Intermediate-acting: NPH (isophane) insulin

4. Long-acting: insulin glargine and insulin detemir

The following table (Table 2) summarizes the time of onset; peak action and duration of action are summarized in the following table.

TABLE 2

| | | | | |
|---|---|---|---|---|
| | | Peak | Effective | Maximum |
| | Onset of | action | duration of | duration |
| Insulin Preparation | Action (h) | (h) | action (h) | (h) |

RAPID-ACTING ANALOGUES AND PREPARATIONS

| | | | | |
|---|---|---|---|---|
| Insulin lispro (Humalog), | ¼-½ | ½-1¼ | 3-4 | 4-6 |
| Insulin | | | | |
| aspart (NovoLog), | | | | |
| Insulin glulisine (Apidra) | | | | |

SHORT-ACTING

| | | | | |
|---|---|---|---|---|
| Regular (soluble) | ½-1 | 2-3 | 3-6 | 6-8 |

INTERMEDIATE-ACTING

| | | | | |
|---|---|---|---|---|
| NPH (isophane) | 2-4 | 6-10 | 10-16 | 14-18 |

LONG-ACTING ANALOGUE

| | | | | |
|---|---|---|---|---|
| Insulin glargine (Lantus) | 3-4 | 8-16 | 18-20 | 20-24 |
| Insulin detemir | 3-4 | 6-8 | 14 | ~20 |
| (Levemir) | | | | |

Methods and Examples of Use Present Invention for Treatment of Glaucoma, Elevated Intraocular Pressure, and Retinal Damage.

Preparation of the patients and helper: To apply eye drops of the therapeutic agents, wash hands with mild antiseptic soap. Be careful not to touch the dropper tip or let it touch your eye lids to avoid contamination. Tilt the head back, or lay down with head extended on a neck pillow; gaze upward and backwards; and pull down the lower eyelid to expose the conjunctival fornix. Place dropper directly over eye away from the cornea and instill the prescribed number of drops. Look downward and gently close your eye for 1 to 2 minutes. Try not to blink and do not rub the eye. Do not rinse the dropper unless one knows how to sterilize in hot water. If other therapeutic, pharmaceutical, biochemical and biological agents or compounds are to be selected to treat the condition with our invention; wait at least 3-5 minutes before using selected antiglaucoma medication or other kinds of ophthalmic medicaments. It is important to instill medications regularly and exactly as prescribed to control IOP and glaucoma. Consult your doctor and/or pharmacist if the systemic medications you are taking together are safe to use with eye drops described.

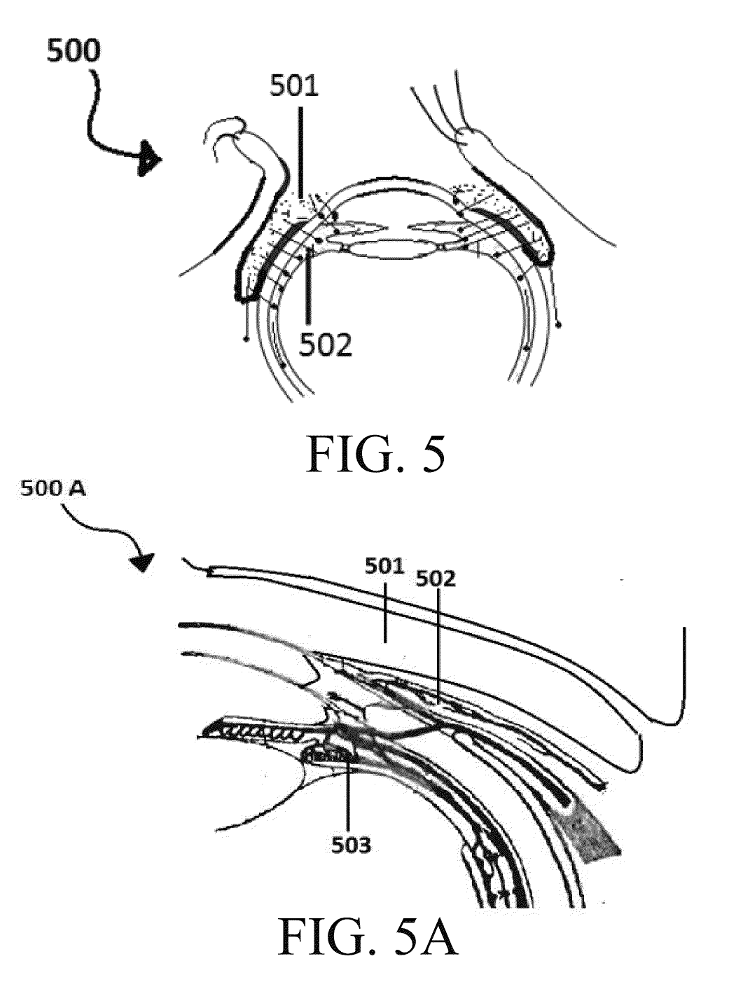

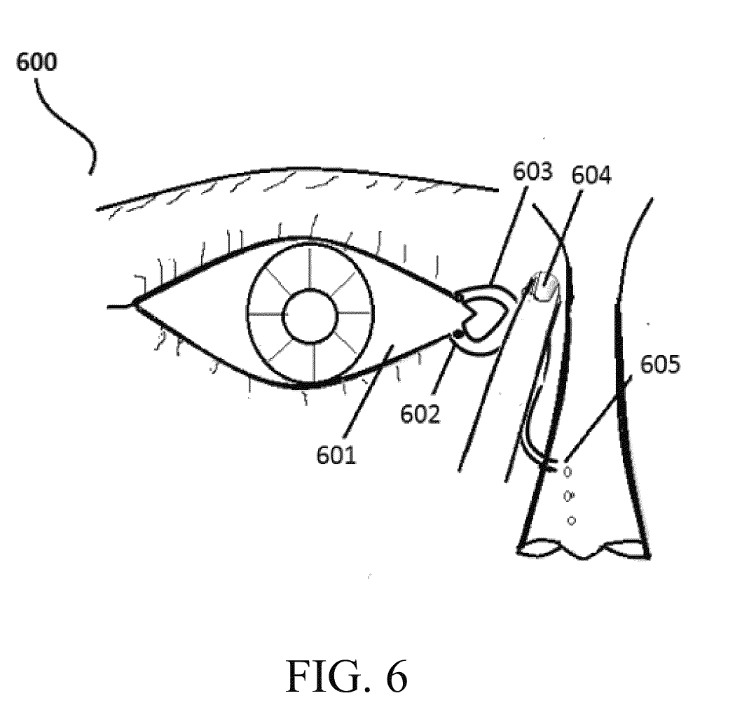

To minimize absorption into the bloodstream and maximize the amount of drug absorbed in the eye, close your eye for one to five minutes after administering the drops and press your index finger lightly against the inferior nasal corner of your eyelid to close the tear duct which drains into the nose (FIGS. 5, 6). This will prevent any adverse systemic effects due to nasal vascular uptake to systemic circulation from the nasolacrimal duct delivery therapeutic agents from the conjunctival sac. Eye drops may cause an uncomfortable burning or stinging sensation which should last for only a few seconds. The anti glaucoma drops take effect after 30-45 minutes. It may take 4-8 hours for complete return of normal vision and pupil size Miotics are used. This process can be repeated every 6-12 or 24 hours for 3-7 days a week till the desirable results are obtained. Once the objective is achieved, use the method once a day before going to bed to keep the glaucoma under control besides reducing the dose. The therapeutic agents are dropped to the conjunctival sac as shown in the FIGS. 5, 6.

Our studies also found that local application of rapid action or other types of insulin formulations on the balding scalp, eye lid hair line, oral and nasal mucosa, and conjunctival sac did not change the blood sugar (no hypoglycemic effects) levels indicating that it is safe to use up to 3-5 IU insulin to the conjunctival sac of both eyes. The typical threshold for hypoglycemia is 70 mg/dL (blood sugar level of 3.9 mmol/L), although it may be higher or lower depending on a patient's individual blood glucose target range. Signs and Symptoms of hypoglycemia include erratic heartbeat, sweating, dizziness, confusion, unexplained fatigue, shakiness, hunger, feeling of heat, and potential loss of consciousness. Once symptoms of hypoglycemia develop, it should be treated immediately with oral ingestion of a fast-acting carbohydrate such as glucose tablets, fruit juice, fruit bowel, chocolate bar, or regular Coca-Cola, sugary drinks or eat plain sugar followed with a drink of water.

Preparation of Insulin Drops

Take 100 units of rapid or intermediate acting insulin (or IGF-1) and dilute in 10 ml of sterile saline or distilled water or other carriers and facilitators as described above. The pH can be adjusted to prevent the sting when dropped to the conjunctival sac. Nanograms or micrograms of local anesthetics may be added to prevent stinging. In this preparation each ml contains 10 units of insulin. In pharmacies, a drop used to be another name for a minim, which would make it 0.0616 milliliters. But now the drop is standardized in the metric system to equal exactly 0.05 milliliters. That is 20 drops make one ml (cc). That means each drop contains 0.5 units of insulin. The concentration of the insulin content can be increased to 0.75, 1.00, 1.5, or 2.00 units of insulin per drop by increasing the insulin content in the dilutant preparation. It can be also decreased by reducing the insulin units used for the preparation of the ophthalmic drops. Instill one drop to each eye lower lid fornix and/or everted upper eyelid (conjunctival sac) as a single agent. If other combination anti glaucoma agents is to be used, first use insulin drops, wait for 3-5-10 minutes and apply the other therapeutic, pharmaceutical, biochemical and biological agents or compounds. After this procedure, instill one more insulin drop to further enhance the uptake of the other selected therapeutic agents and augment-amplify the effects their activity at cellular level. This step is optional and may not be needed in most of the cases.

The dose used in our invention can be appropriately selected depending upon symptom, age, dosage form, existing health conditions etc. The pH can be within a range which is acceptable to ophthalmic preparations within a range from 4 to 8.

The above pharmaceutical eye drop preparation of our invention may contain antibacterial components which are non-injurious to the eye when used. Examples are: thimerosal, benzalkonium chloride, methyl and propyl paraben, benzyldodecinium bromide, benzyl alcohol, or phenylethanol. Due to autism controversy, we will avoid using thimerosal.

The therapeutic pharmaceutical preparation may also contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The therapeutic, pharmaceutical, biochemical and biological agents or compounds used in our invention may also contain a non-noxious pharmaceutical carrier, or with a non-toxic pharmaceutical inorganic substance. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, olyvinylpyrrolidone, isopropyl myristate and other traditionally acceptable carriers.

The therapeutic preparation may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic. Furthermore, appropriate ophthalmic vehicles can be used as carrier media for the current purpose including conventional phosphate buffer vehicle systems, isotonic boric acid vehicles, isotonic sodium chloride vehicles, isotonic sodium borate vehicles and the like.

The anti glaucoma therapeutic agents preparation may also contain surfactants such as polysorbate surfactants, polyoxyethylene surfactants (BASF Cremaphor), phosphonates, saponins and polyethoxylated castor oils and polyethoxylated castor oils which are commercially available.

The pharmaceutical preparation may too contain wetting agents that are already in used in ophthalmic solutions such as arboxymethylcellulose, hydroxypropyl methylcellulose, glycerin, mannitol, polyvinyl alcohol or hydroxyethylcellulose and the diluting agent may be water, distilled water, sterile water, or artificial tears. The wetting agent is present in an amount of about 0.001% to about 10%.

The ophthalmic formulation of this invention may include acids and bases to adjust the pH; tonicity imparting agents such as sorbitol, glycerin and dextrose; other viscosity imparting agents such as sodium carboxymethylcellulose, polyvinylpyrrdidone, polyvinyl alcohol and other gums; suitable absomtion enhancers, such as surfactants, bileacids; stabilizing agents such as ktioxidants, like bisulfites and ascorbates; metal chelating agents, such as sodium EDTA; and drug solubility enhancers, such as polyethylene glycols. These additional ingredients help make commercial solutions with stability so that they need not be compounded as needed.

Ophthalmic medications compositions will be formulated so as to be compatible with the eye and/or contact lenses. The eye drop preparation should be isotonic with blood. As will be the ophthalmic compositions intended for direct application to the eye will be formulated so as to have a pH and tonicity which are compatible with the eye. This will normally require a buffer to maintain the pH of the composition at or near physiologic pH (i.e., 7.4) and may require a tonicity agent to bring the osmolality of the composition to a level at or near 210-320 millimoles per kilogram (mOsm/kg).

The FIGS. 1 to 7 show the histology of the eye, site of production of aqueous humor, its circulation in the posterior and anterior chambers, the structures implicated in pathogenesis of glaucoma; eye structures involved in the entry and site of action of various therapeutic, pharmaceutical, biochemical and biological agents or compounds, and how to prevent systemic effect by simple method of pressing on the lacrimal duct system FIG. 6.

FIG. 1 is the drawing of the longitudinal section of the eyeball 100, including the optic nerve 120 dura mater 101, leptomeninges, and sclera, choroid, ciliary body, iris and cornea are drawn to show their relationship with each other, trabecular meshwork and aqueous humor circulation. The dura mater 101 covering of the optic nerve 120 continues with the sclera 106 and cornea. The Pia 104 and Arachnoid 102 mater forms the sub arachnoid space 103 containing CSF with arachnoid villi formation 135 on the arachnoid mater. When the CSF pressure rose in the subarachnoid space which reflects the CSF pressure of the CNS can be transmitted to the anterior chamber to raise the aqueous humor pressure.

Pia and arachnoid continue through the lamina cribrosa 105 as choroid with formation of supra choroidal and inter choroidal spaces 107. The choroid 107, an extension of the pia-arachnoid mater continues to cover ciliary muscle 108, non pigmented cells of the iris stroma 111, and various forms of trabecular meshwork 110 which drains the aqueous humor; to iris-scleral angle and Canal of Schlemm 109 and corneal endothelium 112. Note the arachnoid villi 135 projecting from the subarachnoid space into dura and close to BV. All these structures especially 107 to 112 plays a role aqueous humor dynamics, in pathology of glaucoma; insulin and other known therapeutic agents action of our invention against ocular pathology at these sites as described below (from Shantha T R and Bourne G H. Arachnoid villi in the optic nerve of man and monkey. Expt Eye Res 3:31-35 (1964). Acta Anat 61:379-398 (1965).

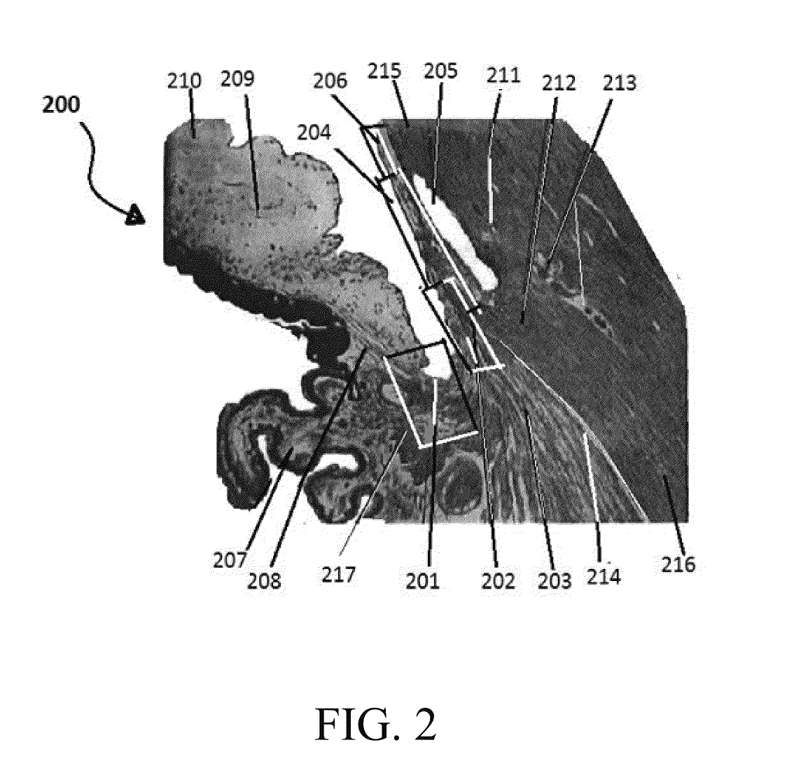

FIG. 2 is the histological longitudinal section of the eye 200 showing the structures involved in the production and drainage of aqueous humor and possible site of action of therapeutic agents in the treatment of glaucoma and related diseases. The important structure involved in drainage of aqueous humor from the anterior chamber are: The uveoscleral or nonconventional Inner uveal meshwork 201, Corneoscleral meshwork 202, juxtacanalicular or cribriform trabecular meshwork 204, Schlemm's canal 205, Corneal endothelium joining the trabecular meshwork 206, Longitudinal and circular fibers of the ciliary muscles 203, 217, muscle fibers of the iris 208, 209, iris 210, Scleral sinus vein 211, Scleral Spur 212, Scleral Veins 213, Suprachoroidal space between choroid and sclera 214. The cornea 215 and sclera 216 do not participate in aqueous humor circulation (Modified from Grays Anatomy).

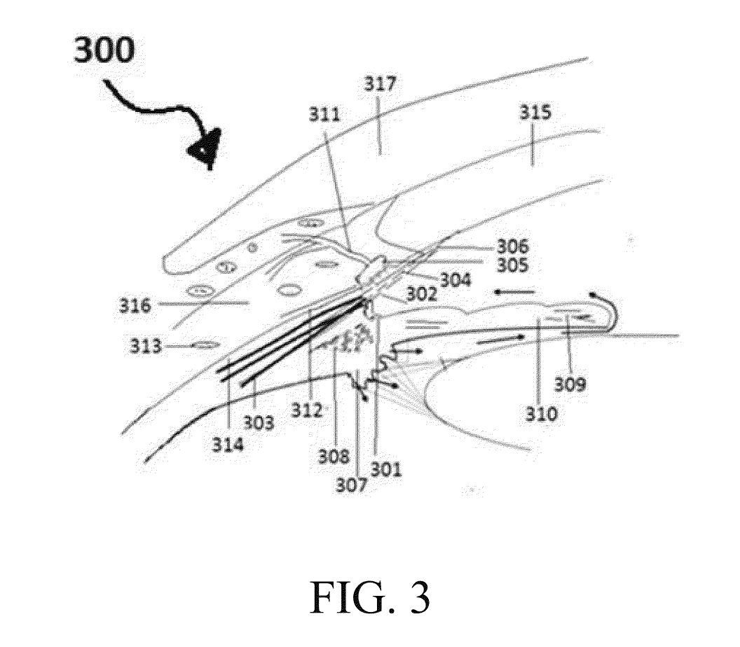

FIG. 3 is the drawing of the longitudinal section of the eye 300 showing the structures similar to FIG. 2 involved in the production and drainage of aqueous humor and possible site of action of therapeutic agents in the treatment of glaucoma and related diseases in our invention. The structure involved in drainage of aqueous humor from the anterior chamber are: The uveoscleral or nonconventional Inner uveal meshwork 301, Corneoscleral meshwork 302, Juxtacanalicular or cribriform trabecular meshwork 304, Schlemm's canal 305, Corneal endothelium joining the trabecular meshwork 306, Longitudinal 303, and circular 308 fibers of the ciliary muscles; muscle fibers of the iris 309, iris 310, Scleral sinus vein 311, Scleral Spur 312, Scleral Veins 313, Suprachoroidal space between choroid and sclera 314.

The cornea 315 and sclera 316 do not participate in aqueous humor circulation. The conjunctival sac (fornix) where the therapeutic, pharmaceutical, biochemical and biological agents or compounds are deposited to be transported to the ciliary body and irido-scleral angle structures which play a role in reducing the IOP by decreasing the aqueous humor production and/or increasing trabecular meshwork out flow (From Shantha T R and Bourne G H. Some observations on the corneal endothelium. Acta Ophthalmologica 41: 683-688 (1963).

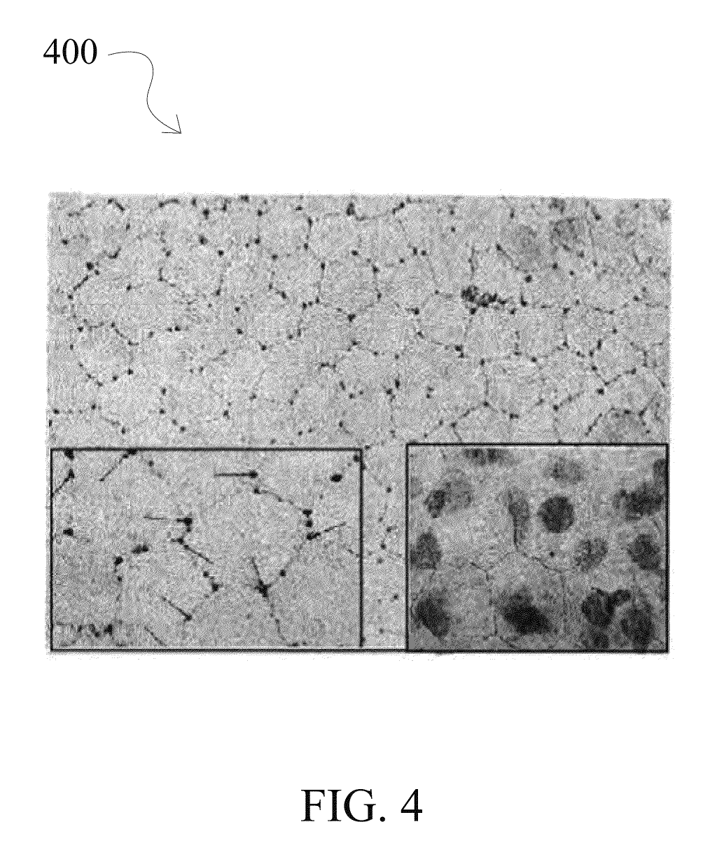

FIG. 4: shows the isolated whole mount of the corneal endothelium 400 treated with 1% aqueous silver nitrate to demonstrate inter cellular junctions. Preparation demonstrates the squamous nature of the corneal endothelium cells dotted with intercellular pores stained deeply which allow aqueous humor to enter the corneal stroma. Left insert is the higher magnification from FIG. 4, dotted with the round or oval openings (arrows) found in between some areas of the margins of two adjacent cells which are capable of letting the aqueous humor permeation to the corneal stroma. Note that the most of the intercellular junctions are tightly bound. Right insert shows the Pigment epithelium from the retina, prepared like corneal endothelium. Note these pigment epithelium of the retina do not demonstrate any areas of pores in between the cells junction as seen in the corneal endothelium to allow any free flow of the choroidal tissue fluids to and from the retina; thus play an important role as retinal-choird-blood barrier (From Shantha T. R and Bourne G. H. Some observations on the corneal endothelium. Acta Ophthalmologica 41: 683-688 (1963).

FIG. 5 is the diagram of the longitudinal section of the eye 500 showing conjunctival sac and the iridocorneal angle involved in the pathophysiology of glaucoma and the absorption of therapeutic agents including therapeutic agents from the present invention, from conjunctival sac through the complex vascular anastomosis (500A), which picks up the therapeutic agents from the conjunctival sac and delivers to the ciliary body, epithelium, muscles, and trabecular meshwork (drum stick markers) enhancing the out flow of aqueous humor from the anterior chamber resulting in reduction of intraocular pressure.

The diagram shows how easy it is for the therapeutic agents to enter the afflicted site from the conjunctival sac. The conjunctival sac 501 where the local insulin and other therapeutic agents are deposited for the treatment of glaucoma. From here the therapeutic agents enter into the anterior chamber, trabecular meshwork, and ciliary body 502 passing through the sub conjunctival area of the eye and their destination to reach the eye structures involved in aqueous humor production and exit. The drum stick markers indicate the site of entry of therapeutic agents exerting their effect exerting their therapeutic effect of lowering the IOP at the site of aqueous humor production and exit.

FIG. 5A digramatic presentation section of the anteriour part of the eye 500A. Note the rich vascular plexus 502 under the conjunctiva of the eye ball which helps to transport the therapeutic agents from the conjunctival sac 501 to various structures 503 involved in glaucoma production and aqueous humor out flow. Note the Rich vascular plexus of the iris, choroid, ciliary body 503, communicating with the sub scleral vascular and choroidal net work which delivers insulin and anti glaucoma therapeutic agents to various structures between the ciliary body and the iridoslceral angle and scleral—corneal space, and subscleral network of vascular plexus. This diagram shows the rich vascular net work to deliver the insulin and therapeutic agents and to reduce the IOP and treat glaucoma.

FIG. 6 is the diagramatic presentation 600 showing the route of drainage of the lacrimal fluid and therapeutic agents from the conjunctival fornix 601 to the nasal mucosa 605 and method to prevent it. A simple method of applying the finger pressure at the medial eye agle and nasal junction, the location of the lacrimal punctum, canaliculi 602, 603 and lacrimal sac with a finger 604 will prevent the therapeutic agents flow to the nose, and its contact with the nasal mucosa 605, and their associated systemic adverse effects.

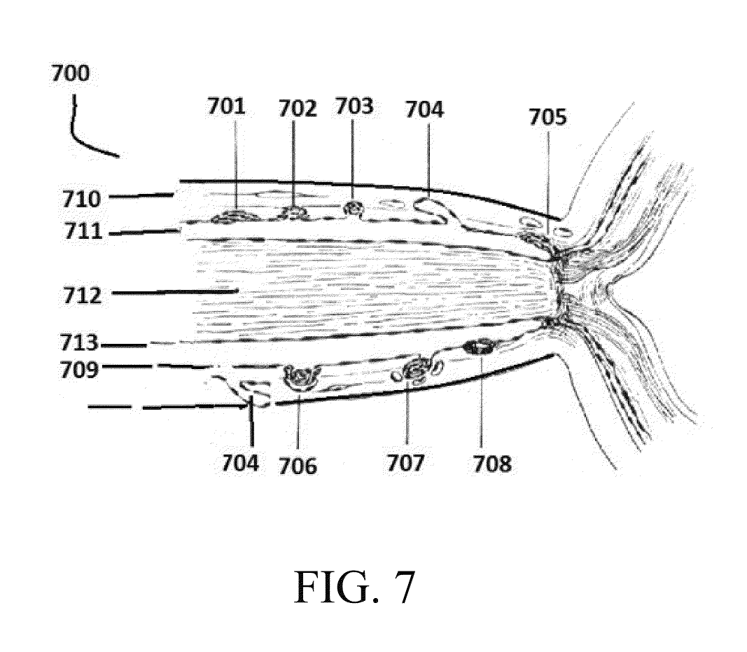

FIG. 7 is the diagramatic presentation 700 showing the lognitudinal section of the optic nerve 120 and the posterior part of eye ball. It shows the dural covering 701, arachnoid mater 702 with formation of mulitile aracahnoid villi of different type 735 penetrating dura and some of them opening and siturated close to the drainage venous channel like trabecular meshwork to venous cahannel-canal of Schlemm. Arachnoid villi communicate with the subarachnoid space 703. The dural covering 701 continue with the sclerea 706 of the eye, pia-arachnolid after uniting behind the lamina cribrosa 705 continue as choroid 707 of the eye ball. Hence the CSF from the CNS can permeate to choroid and supply neuroptophic factors to retina. It can be mixed with aqueous humor draining through the suprachoroidal space and choraoidal vascular plexus. The raise in CSF pressure can also raise the aqueous humor pressure in the anetrior champber due to blockage of the choriodal flow of aqueous humor and physical trasmission of elevated pressure.

The therapeutic agents agent such as insulin and other neurotrophic factors can easily reach retinal through the choroidal route. Thus they can have therapeutic trophic effect on the disease afflicted retina. Based on our histological findings, there is every likehood that the increased IOP may prevent (due to build-up of back pressure in the choroid due to elevated CSF pressure from the CNS) the neroptrophic factors in the CSF reaching the retinal through the lamina cribrosa and inter choroidal space; thus contributing to the retinal pathology (From Shantha T. R. and Bourne G H. Arachnoid villi in the optic nerve of man and monkey. Expt Eye Res 3:31-35 (1964). Histological and Histochemical studies of the choroid of the eye and its relations to the pia-arachnoid mater of the central nervous system and Perineural epithelium of the peripheral nervous system. Acta Anat 61:379-398 (1965).

The Following are the Examples of Using Our Invention Insulin and/or IGF-1 Growth Factors Alone or in Combination with Known Anti Glaucoma Therapeutic, Pharmaceutical, Biochemical and Biological Agents or Compounds to Treat Glaucoma and Rised IOP

### EXAMPLE 1

Select the patient and establish the type of glaucoma (definite diagnosis) the person is suffering. Record the degree of retinal damage by the thorough examination. Position the patient in supine posture with head extended on a support. Using a dropper, instill one drop of insulin preparation in each eye lower lid fornix and/or everted upper eyelid. Apply slight pressure at the nasal angle of eye pressing on the nasolacrimal canaliculi-sac-duct to prevent leaking of the therapeutic agents to the nose to avoid systemic absorption and its adverse effects (see FIG. 6). Stay still for 3-5 minutes and resume the desired posture.

### EXAMPLE 2

Select the patient and establish the type of glaucoma the person is suffering (definite diagnosis) from. Record the degree of retinal damage by the examination. Position the patient in supine posture with head extended on a support. Using a dropper, instill one drop of IGF-1 preparation in each eye lower lid fornix and/or everted upper eyelid. Apply slight pressure at the nasal angle of eye pressing on the nasolacrimal canaliculi-sac-duct to prevent leaking of the therapeutic agents to the nose. Stay still for 3-5 minutes and resume the natural desired posture.

### EXAMPLE 3

Select the patient and establish the type of glaucoma the person is suffering (definite diagnosis) from. Record the degree of retinal damage by the examination. the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Following this, instill a drop in each afflicted eye Prostaglandin analogs like latanoprost (Xalatan), bimatoprost (Lumigan) and travoprost (Travatan) and Rescula which increase uveoscleral outflow of aqueous humor: Bimatoprost in addition also increases trabecular mesh work outflow. Stay still for 3-5 minutes and resume the natural desired posture. The dose of these medications can be reduced because the insulin has augmentation—amplification effects on these therapeutic agents anti glaucoma effect many times both in their uptake from the extracellular fluid and inside the cell. Insulin also improves the retinal function.

### EXAMPLE 4

Select the patient and establish the type of glaucoma the person is suffering (definite diagnosis). Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of the eye pressing on the nasolacrimal canaliculi-sac-duct to prevent leaking of the therapeutic agents to the nose (FIG. 6). Then instill selected topical beta-adrenergic receptor antagonists such as: Timoptic, Timoptic (XE/GFS, or Ocudose), Betoptic, Optipranolol, Ocupress) timolol, levobunolol (Betagan), and betaxolol. They are either non selective like Timopitc or selective blokcers such as Betopitc decrease aqueous humor production by the ciliary body and ciliary process. Stay still for 3-5 minutes and resume the natural desired posture. The dose of these medications can be reduced because the insulin enhances the anti glaucoma effect many times and improves the retinal function.

### EXAMPLE 5

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Then instill the selected topical Alpha2-adrenergic agonists such as brimonidine (Alphagan, Iopidine, Propine) which act by dual mechanism of decreasing aqueous production and increasing trabecular outflow. Stay still for 3-5 minutes and resume the natural desired posture. The dose of these medications can be reduced because the insulin augments—amplifies the anti glaucoma effect many times and improves the retinal function.

### EXAMPLE 6

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Then instill selected topical a beta2-agonist or Less-selective sympathomimetics like epinephrine and dipivefrin (Propine) which increase outflow of aqueous humor through trabecular meshwork and possibly through uveoscleral outflow pathway. Stay still for 3-5 minutes and resume the natural desired posture. The dose of these medications can be reduced because the insulin enhances the anti glaucoma effect many times (augmentation—amplification effects) and improves the retinal function.

### EXAMPLE 7

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of pressing on the nasolacrimal canaliculi-sac-duct to prevent leaking of the therapeutic agents to the nose. Then instill selected topical Miotic agents like pilocarpine and Ecothiopate (parasympathomimetics is also used in chronic glaucoma). These therapeutic agents cause contraction of the ciliary muscle, tightening the trabecular meshwork; opening the intertrabecular spaces and allowing increased outflow of the aqueous humor. Stay still for 3-5 minutes and resume the natural desired posture. The dose of these medications can be reduced because the insulin enhances the anti glaucoma therapeutic effect many times and improves the retinal function.

### EXAMPLE 8

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Then instill selected topical Carbonic anhydrase inhibitors like dorzolamide (Trusopt), brinzolamide (Azopt), acetazolamide (Diamox). These ocular drops lower secretion of aqueous humor by inhibiting carbonic anhydrase in the ciliary body and process. Stay still for 3-5 minutes and resume the natural desired posture. The dose of these medications can be reduced because the insulin enhances the anti glaucoma therapeutic effect many times and improves the retinal function.

### EXAMPLE 9

Select the patient and establish the type of glaucoma the person is suffering (definite diagnosis) from. Record the degree of retinal damage by the examination. Advise the patient to take Oral carbonic anhydrase inhibitors such as Diamox, Neptazane or any other oral anti glaucoma therapeutic agents available 30 minutes before insulin ophthalmic drops. Position the patient in supine posture with head extended on a support. Using a dropper, instill one drop of insulin preparation in each eye lower lid fornix and/or everted upper eyelid. Then apply slight pressure at the nasal angle of the eye pressing on the nasolacrimal canaliculi-sac-duct to prevent leaking of the therapeutic agents to the nose and then assume the desired posture.

### EXAMPLE 10

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Then instill selected topical Physostigmine which is also used to treat glaucoma and delayed gastric emptying. Instillation of 0.25% physostigmine sulphate eye drops that causes a sustained miosis. Stay still for 3-5 minutes and resume the natural desired posture. The dose of these medications can be reduced because the insulin enhances the anti glaucoma therapeutic effect many times and improves the retinal function.

### EXAMPLE 11

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Then instill selected topical combination eye drops such as Timoptic and Trusopt (Cosopt), Xalatan and Timoptic as well as Alphagan and Timoptic. Combination eye drops may improve compliance if more than one drug is needed to control the eye pressure as it is more convenient to deal with just instill one bottle. Stay still for 3-5 minutes and resume the natural desired posture. The dose of these medications can be reduced because the insulin enhances the anti glaucoma therapeutic effect many times and improves the retinal function.

### EXAMPLE 12

Select the patient and establish the type of glaucoma the person is suffering (definite diagnosis) from. Record the degree of retinal damage by the examination. Position the patient in supine posture with head extended on a support. Using a dropper, instill one drop of insulin preparation in each eye lower lid fornix and/or everted upper eyelid. Then apply slight pressure at the nasal angle of eye pressing on the nasolacrimal canaliculi-sac-duct to prevent leaking of the therapeutic agents to the nose. Then instill selected topical combination eye drops which contains insulin and any of the above suitable anti glaucoma medication such as Prostaglandin analogs like latanoprost (Xalatan), bimatoprost (Lumigan) and travoprost (Travatan) and Rescula; Topical beta-adrenergic receptor antagonists such as: Timoptic, Timoptic (XE/GFS, or Ocudose), Betoptic, Optipranolol, Ocupress) timolol, levobunolol (Betagan), and betaxolol (They are either non selective like Timopitc or selective blokcers such as Betopitc); Alpha2-adrenergic agonists such as brimonidine (Alphagan, Iopidine, Propine); sympathomimetics like epinephrine and dipivefrin (Propine); parasympathomimetics like pilocarpine and Ecothiopate; Carbonic anhydrase inhibitors like dorzolamide (Trusopt), brinzolamide (Azopt), acetazolamide (Diamox); Physostigmine used for sustained miosis and any other anti glaucoma agents in the developments.

The following table, Table 3, uses trade names and summarizes the popular glaucoma medications and their site of action which can be combined with insulin which makes them more effective. When insulin is used, reduce the dose of the above and the following medications because the insulin has augmentation—amplification effects on anti glaucoma therapeutic, pharmaceutical, biochemical and biological agents or compounds.

TABLE 3

| | | | Increase |
|---|---|---|---|
| | Reduce aqueous | Increase Trabecular | Uveoscleral |
| | humor production | outflow | outflow |
| A | Timoptic | Lumagan | Xalatan |
| B. | Truspot | Pilocarpine | Alphagan |
| C | Alphgan | | |

Combination eye drops: Alphagan and Timoptic; and Xalatan and Timoptic

The combination anti glaucoma medications are already in existence (available in the pharmacy) such as: Timoptic, Trusopt (Cosopt), Xalatan, Timoptic, Alphagan and Timoptic. Combination eye drops containing insulin and appropriate anti glaucoma therapeutic agents may improve compliance if one has to instill one drug needed to control the eye pressure as it is more convenient to deal with just instill one bottle. Further, the dose of the anti glaucoma agents which are already in use can be reduced to avoid the local and systemic complications and the cost of the pharmacological agents. The dose, and cost of these medications can be reduced because the insulin enhances the anti glaucoma therapeutic effect many times and improves the retinal function.

### EXAMPLE 13

U.S. Pat. No. 6,482,854 B1 discloses that the elevated glutamate levels are associated with glaucoma, and damage to retinal ganglion cells. This damage can be controlled by a compound capable of reducing glutamate induced excitotoxicity in a concentration effective to cause reduction of such excitotoxicity by administering systemically or as ophthalmic drops. Such a compound that is an antagonist of NMDA receptor-mediated excitotoxicity, in a concentration effective to cause reduction of said excitotoxicity, said antagonist being capable of crossing the blood brain barrier and the blood retina barrier.

They list dozens of such compounds for use. The damage to the retina is blamed on these NMDA and their associated receptors by glutamate in the retina. A number of antagonists to the NMDA receptor are antinociceptive in animal models but are associated with significant dose-limiting side effects. Such dose limiting effect can be overcome with combination of Insulin drops, which reduces the dose of NMDA receptor antagonist.

Commercially available NMDA-receptor antagonists include ketamine, dextromethorphan, memantine, and amantadine. The opioids, methadone, dextropropoxyphene, and ketobemidone are also antagonists at the NMDA receptor. We have prepared ketamine ophthalmic drops with insulin on dogs with no untoward effect. Combining insulin with suitable NMDA receptor antagonist can be effectively used to control glaucoma and at the same time protect retina from the cytotoxic effects of glutamate.

It is important to note that the ketamine has local anesthetic effects in addition. Combination of ketamine and insulin can be an effective therapy to prevent or curtail the retinal damage due to glutamate damage by blocking NMDA receptors. At the same time control the glaucoma and take away the sting felt when eye drops are instilled.

### EXAMPLE 15

Primary open-angle glaucoma (POAG) is a progressive disease leading to optic nerve damage and, ultimately, loss of vision. The cause of this disease has been studied for decades. Glaucoma results in the neuronal degeneration of the retina and optic nerve head, a gradual loss of retinal ganglion cells (RGC), with a decline of visual function, leading to blindness (Clark et al., Nature Reviews Drug Discovery, 2003, Vol. 2(6):448-4591). One theory besides many others suggests that raised IOP, may be tied with genetic defects on the optic nerve head, due to axonal transport along the optic nerve leading to RGC injury. It is thought that the disturbance of axonal transport of the optic nerve hinders traffic of intracellular molecules between the RGC soma and its terminal. Besides axonal transport, the membranes of the optic nerve, choroid, and the BV of the eye do communicate with each other thus transporting the protective or disease afflicting agents to the retina and the rest of the eye (Shantha T R and Bourne G H: Arachnoid villi in the optic nerve of man and monkey. Experimental Eye Research, 3:31-35 (1964). Shantha T R: The Relationship of Retro bulbar Local Anesthetic Spread to the Neural Membranes of the Eyeball, Optic Nerve and Arachnoid Villi in the Optic Nerve. Anesthesiology 73(No 3A):A850 (1990). Shantha T R and Bourne G H: The “PERINEURAL EPITHELIUM”: A new concept. Its role in the integrity of the peripheral nervous system. In Structure and Function of Nervous Tissues. I. (G H Bourne, Ed.). Academic Press, New York. 1968. pp 379-459. Shantha T R and Bourne G H: Histological and histochemical studies of the choroid of the eye and its relations to the pia-arachnoid mater of the central nervous system and Perineural epithelium of the peripheral nervous system. Acta Anat 61:379-398 (1965)). Among the intracellular molecules of importance are neurotrophic factor peptides molecules from CSF transported through the choroid to the retina; which stimulate or otherwise maintain growth and integrity of retinal neural tissue. The transport of neurotrophic factors from the brain to the cell body of RGC is essential to the survival of the RGC. Deprivation of neurotrophic factors can induce apoptosis of neurons, and may be a cause of glaucoma-induced RGC apoptosis. The neurotrophin (“NT”) family of peptides includes nerve growth factor (NGF), brain-derived neurotrophic NT-415, whereas TrkC is selective for NT-3. After binding, the NT-receptor complex is internalized and transported via the axon to the RGC soma. These receptors undergo ligand induced phosphorylation and dimerization, and activate a cascade of Ras protein-mediated signal transduction events that affect multiple vital functions of the neuron.

Thus, these receptors play a fundamental role in the regulation of survival and differentiation of developing neurons and contribute to the maintenance of neuronal function in adult life. Even today, the underlying causes of glaucoma are not well understood. Why the glaucoma is characterized by damage to the optic nerve, accompanied by a decrease in the normal visual field is still unknown? One early warning sign of raised IOP is visual field loss. Raised IOP is blamed on this loss. Research shows, it is more than this glaucoma physical etiology. Administration of insulin as described in this invention with selected neurotophic factors can effectively stop, curtail or cure vision loss due to degeneration of ganglion cells and progression of retinal functional degeneration associated with glaucoma.

U.S. PATENT APPLICATION PUB. NO.: 2001/0047012 A1. Disclose treating glaucoma and glutamate excitotoxicity on ganglion cells administering one glutamate antagonist and at one intraocular pressure-lowering agent. The method of claim wherein the glutamate antagonist is memantine, or eliprodil or their own synthetic formula. Whereas the IOP-lowering agent is selected from the group consisting of a adrenergic agonists, β blockers, prostaglandins, carbonic anhydrase inhibitors, betaxolol or S-betaxolol, brirnonidine, latanoprost, travoprost, and timolol. Combining ophthalmic drops consisting of anti glutamate therapeutic agents and suitable known anti glaucoma pharmaceutical preparation with insulin eye drops can be very effective in controlling the glaucoma and the preventing further retinal damage.

### EXAMPLE 16

A method and a composition for treating or reducing and/or controlling intraocular pressure by administering an effective amount of a renin inhibiting compound of the formula; wherein the renin inhibiting compound is administered in combination with a beta-adrenergic antagonist, a steroidal anti-inflammatory agent or an angiotensin converting enzyme inhibiting and angiotensin converting enzyme receptor blocker compound. These therapeutic agents will be more effective when used with insulin primed eyes as described in our invention.

### EXAMPLE 17

U.S. PATENT APPLICATION PUB. NO.: 2009/0082338 A1 discloses glaucoma preventive or a remedy for ocular hypertension, with a potent ocular hypotensive effect and prolonged duration thereof. A preventive or a remedy for glaucoma comprising a Rho kinase inhibitor and a α₁ blocker in combination is described. The therapeutic agents effectiveness of this mode of therapy is augmented by the use of insulin ophthalmic drops before and after the application of the above describe new inventive method.

### EXAMPLE 18

There are several different glaucoma drainage surgical implants developed to drain the aqueous humor. These include the original Molteno, the Baerveldt tube shunt, or the valved implants (Ahmed glaucoma valve implant or the ExPress Mini Shunt, pressure ridge Molteno implants). The main disadvantage of these implants is: The scarring over the conjunctival dissipation segment of the shunt may become too thick for the aqueous humor to filter through which may require preventive measures using additional surgery. Our invention of using insulin with anti fibrotic medications such as 5-Fu, or mitomycin ophthalmic drops will result reduction or elimination of this formation of obstructive fibrous tissue, and keep open the shunt for long period of time to relieve the IOP. The dose of these chemotherapeutic agents can be reduced due to augmentation (amplification effects) of their activity many folds by insulin (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228)

### EXAMPLE 19

Use of Chelation Therapeutic Agents:

It is a known fact that the trabecular meshwork gets deposits of protenacious complexes in glaucoma. It is highly likely; they do have many metallic and organic deposits such as iron and calcium in them due to death of trabecular meshwork cells and protenacious deposits. Chelation therapy with Ethylenediaminetetraacetic acid (EDTA), Methylsulfonylmethane (MSM), and Deferoxamine (also known as desferrioxamine B, desferoxamine B, DFO-B, DFOA, DFB or desferal) will clear the clogged mesh work. They remove any metal, calcium, and other metallic as well as protenacious deposits in the trabecular meshwork and the lens which affect their physiological role in the proper functioning of the eye.

The EDTA unclogs blood vessels; controls free radical damage due to lipid peroxidation by serving as a powerful antioxidant; increases tissue flexibility by uncoupling age-related cross-linkages that are responsible for loss of trabecular meshwork and lens tone; removes lead, cadmium, aluminum, and other metals, restoring enzyme systems to their proper functions; enhances the integrity of cellular and mitochondrial membranes; reduces the tendency of platelets to cause coagulation too readily which can clog the trabecular meshwork; unclogs the clogged draining vascular system, increases tissue flexibility by uncoupling age-related cross-linkages (age related glycation) that are responsible for loss of trabecular meshwork and lens tone. The cataract being a calcium deposit in the lens, the use of EDTA along with insulin as described in our invention can slow down, arrest or reverse the cataract process.

Deferoxamine is a chelating agent used to remove excess iron from the body. By removing excess iron, it reduces the damage done to various organs and tissues, such as the liver, CNS, eyes, trabecular meshwork and retina. The damage we see in retina can be due to excessive iron form the choroid and retinal blood vessels leaking excessive iron. The role of iron (metallobiology) in neurodegenerative disorders has long been implicated, with particular attention given to iron as it is one of the most important redox metals, which have been largely linked to senile toxicity and neurodegenerative disorders such as Alzheimer's and Parkinson's diseases and aging patients (Stankiewicz J M, Brass S D (2009) Role of iron in neurotoxicity: a cause for concern in the elderly? Curr Opin Clin Nutr Metab Care 12:22-9).

The redox switching capability of iron from ferrous to ferric state, and vice versa, makes it one of the most dangerous catalytic elements responsible for the neurodegenerative process. Iron generates free radicals and reactive oxygen species in the aged nerve tissue as evidenced by higher heme oxygenase-I, which contributes to increased susceptibility to oxidative stress in older people (Hirose W, Ikematsu K, Tsuda R (2003) Age-associated increase in heme oxygenase-1 and ferritin immunoreactivity in the autopsied brain. Leg Med 5(Suppl. 1):360-6). Retina is nerve tissue and is not spared by the neurodegenerative process by iron.

Biochemical events surrounding iron-mediated catalytic events, which give rise to oxidative stress and free radical generation, can be described as known Fenton reaction as indicated below.

Fe3+.O²→KO2-Fe2⁺+O₂ (Step I); Fe²++H₂O₂—Fe³⁺+OH—+KOH (Step II)

### Combining Step I and II: .O2⁻+H₂O₂→.HO⁻+O2

The role of iron in the neurodegenerative process can be best described in three distinct phases: accumulation, invasion, and catalytic activity. A recent study also shows that it speeds healing of nerve damage (and minimizes the extent of recent nerve trauma), hence the retinal damage in glaucoma can be reduced or curtailed by iron Chelation. Deferoxamine may modulate expression and release of inflammatory mediators as indicated in Fenton reaction by specific cell types thus reduce or stop the damage to the trabecular meshwork, lens and retina.

Methylsulfonylmethane (MSM) is a supplement form of sulfur that is found in our living tissues. MSM supports healthy connective tissues like tendons, ligaments, and muscle. Hence it is important to bring back the trabecular meshwork and ciliary body with its muscle and tendon. MSM makes cell walls permeable, allowing water and nutrients to freely flow into cells and allowing wastes and toxins to properly flow out. MSM is an anti-oxidant that helps to clean the blood stream and flush toxins trapped in our cells. It is also a foreign protein and free radical scavenger which is needed in the treatment of glaucoma. The body uses MSM along with Vitamin C to create new, healthy cells, and MSM provides the flexible bond between the cells.

We prepare the following eye drops containing: 1. EDTA, 2. Deferoxamine, 3. MSM with added preservatives, anti bacterial and DMSO, combined with insulin in proper concentrations. These eye drops are used before or after insulin drops a prophylactic and therapeutic agents for glaucoma to reduce the retinal damage and lower the IOP. These therapeutic agents along with Alagebrium combination with insulin can be used to prevent curtail and/or cure cataract.

### EXAMPLE 21

Diabetes is a major contributor to the development of visual disability and blindness. Glaucoma, retinopathy, and cataract are progressive, degenerative diseases and are the leading causes of blindness in diabetics. Primary open angle glaucoma itself occurs in approximately 4% of diabetics compared to 1.8% of the general population. This increase in intraocular pressure is caused by impairment of the drainage of fluid through the trabecular meshwork due to physical obstruction or restriction of the movement of proteins through this sieving system.

Often, such impairment is due to advanced glycation end products (AGE's); a permanent carbohydrate structures that form when carbohydrates bind to proteins, lipids, or DNA, molecules which are integral to the architecture of tissues and organs and to their maintenance. AGE's form deposits on the endothelial cell membranes of the trabecular meshwork, thereby blocking the movement of the aqueous humor. Overall, AGE's is pathognomic of diabetes, and their deposition is largely responsible for the prevalence of glaucoma, retinopathy, and cataract in diabetic population. Diabetic complications within the eye as the manifestation of type a type IV diabetic of the eye; whereas Alzheimer's disease is a manifestation of type III diabetes of the CNS. So far, there is no established method to reverse the deposition of AGE's.

The only drug under development known to reverse this process is Alagebrium (formerly known as ALT-711), a synthetic chemical developed by Alteon Corporation (now Synvista Therapeutics, Inc.) which is described by the formula 4,5-dimethyl-3-(2-oxo-2-phenylethyl)-thiazolium chloride. Experiments show that its systemic use has a wide margin of safety. Hence, ophthalmic drops containing a measured concentration of Alagebrium plus insulin (as described in our invention) can be used topically to prevent, slow the progression of, or even reverse glaucoma, diabetic retinopathy, and cataract formation without any adverse effects. If the glaucoma is severe, low doses of known therapeutic agents such as prostaglandins can be added to these Alagebrium/insulin eye drops.

### EXAMPLE 22

Trachoma (Ancient Greek: “rough eye”) is an infectious eye disease. It is the leading cause of the world's infection related blindness; affecting 84 million people over the globe and nearly 8 million people are visually impaired and disabled as a result of this disease. At present, most victims of trachoma live in underdeveloped and poverty-stricken countries in Africa, the Middle East, and Asia who cannot afford or uneducated about the seriousness of the condition. Chlamydia trachomatis is the offender and it is spread by direct contact with eye, nose, and throat secretions from affected individuals, or contact with fomites (inanimate objects), such as towels and/or washcloths, that have had similar contact with these secretions.

Flies can also be a route of mechanical transmission. Chlamydia trachomatis organism is a bacterium. It causes both chlamydia trochomatis and lymphogranuloma venereum infections. Though it is a bacterium, it is a virus-like, because it is dependent on nutrition and energy molecules from its host organism to reproduce and cannot survive without humans, because it needs ATP, an energy molecule to reproduce. Trachomatis is as an obligate (can't survive without) intracellular (living inside cells) parasite (where it takes but does not give back).

Chlamydia travels between cells, and between people, in the form a small, dense, spore-like, metabolically-inactive structure, almost like a virus infectious agent but in inactive form. When it enters a host cell, it changes into a reticulate body and uses supplies from the host cell to make copies of itself inside the cell. These reticulate bodies can grow, divide, and metabolize, but they're not infectious. Infections can persist in this manner for a while, or, once there are enough copies, the reticulate bodies can turn back into elementary bodies, burst the cell open, and escape to infect new cells or new people.

Chlamydia causes conjunctivitis, or irritation similar to “pink eye. Untreated, repeated trachoma infections result in entropion—a painful form of permanent blindness when the eyelids turn inward, causing the eyelashes to scratch the cornea. Children are the most susceptible to infection due to their tendency to easily get dirty, but the blinding effects or more severe symptoms are often not felt until adulthood.

The conjunctival inflammation (active trachoma) is usually seen in children; especially preschool children characterized by white lumps in the undersurface of the upper eye lid (conjunctival follicles or lymphoid germinal centers) and by non-specific inflammation and thickening often associated with papillae. Follicles may also appear at the junction of the cornea and the sclera (limbal follicles). Bacterial secondary infection may occur and cause a purulent discharge. If not treated properly with oral and local antibiotics, the symptoms may escalate and cause blindness due to corneal ulceration and scarring.

Sulfonamides oral and drops are effective in treating this condition (Thygeson P (1939). “The Treatment of Trachoma with Sulfanilamide: A Report of 28 Cases”. Trans Am Ophthalmol Soc 37: 395-403). One single oral dose of 20 mg/kg or topical tetracycline (one percent eye ointment twice a day for six weeks is the standard therapy) are used effectively. Azithromycin is preferred because it is used as a single oral dose. Surgery may also be necessary to fix eyelid deformities. Use of Insulin with selected antimicrobial eye drops can be effective in eliminating the eye infection with fewer eye drop instillations instead of using the eye drops for weeks and months.

### EXAMPLE 23

Antiviral agents used to treat herpes zoster of the eye are acyclovir, valacyclovir, and famciclovir taken orally. These agents interfere with DNA synthesis and inhibit viral replication. Topical steroids may exacerbate spontaneous recurrences and occasionally, steroids may be prescribed to reduce inflammation. Topical trifluridine (Viroptic), which inhibits DNA synthesis, is as eye drop 2-3 times a day. Atropine Eye drops are used to keep the pupil dilated, to help prevent a severe form of glaucoma, and to relieve pain.

Our invention will help to the orally administered antiviral agents. The insulin drops are used 1-2 hours after oral intake antiviral agent (by the time they reach the high therapeutic concentration in the blood and tissue fluids), so that the insulin will facilitate the entry of antiviral agents inside the cell and become very effective in lowering or reducing the viral multiplication and viral load in the cells and reduce the viral related eye tissue damage. It can be combined with very small doses of lidocaine if there is pain and local antiviral eye drops. Insulin drops can be used 2-3 times a day on the affected eye in conjunction oral and local and/or antiviral therapy.

### EXAMPLE 24

Use of antioxidant along with insulin can have profound effect in saving the retinal ganglion cells and maintain the proper functioning of the trabecular meshwork. At present the examples of pharmaceutically-acceptable antioxidants include: (1) water soluble antioxidants, such as ascorbic acid, curcumin, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediaminetetraacetic acid (EDTA), sorbitol, tartaric acid, phosphoric acid, and the like.

We have used curcumin diluted in the normal saline from 1 to 5 percent after extracting it from turmeric using solvents such as DMSO and alcohol as well other known solvents. It is very effective in improving the retinal damage due to glaucoma or otherwise. It can discolor the cloth, hence, used absorbent sterile towels to remove the excess drainage from the eye. The sclera can be stained yellow that can be mistaken for jaundice. All the above antioxidants can be used with insulin as described above and there is no systemic reaction.

### EXAMPLE 25

In a prospective study, antibodies to Helicobacter pylori (which cause peptic ulcer and stomach cancer) were found to be significantly higher in people with primary open-angle and exfoliation glaucoma compared to cataract surgery patients. It is supposed that this bacteria may play a role in the pathobiology of these forms of glaucoma as reported in U.S. Patent Application Publication Number 2007/0092592 A1. These inventors also report applying to the eye of said person, an effective amount of an antibacterial agent having activity against the Helicobacter Pylori bacteria to thereby eradicate, inhibit and/or control said bacteria, such as combination of lactoperoxidase and a peroxide donor (rifabutin) and a therapeutically effective amount of a second antibiotic or antimicrobial agent selected from the group consisting of amoxicillin, tetracycline and bismuth compounds.

A proton pump inhibitor is selected from the group consisting of omeprazole, pantoprazole, rabeprazole and lansoprazole; protease, and an antibacterial agent (pronase, trypsin, a-chymotrypsin, serrapeptase, bromelain and pepsin and said antibacterial agent is selected from the group consisting of an antibiotic, an anti-protozoan drug and a bismuth preparation). An antibiotic is selected from the group consisting of amoxicillin, erythromycin and clindamycin said anti-protozoan drug is selected from the group consisting of metronidazole and tinidazole and said bismuth preparation or quinoline compound is selected from the group consisting of compounds represented by the formula.

Local antibiotics should be carefully selected; those which do not cause local allergic condition. Using insulin drops with any of the safe ophthalmic antibiotic eye drops. Insulin not only increased the therapeutic agent's effectiveness against the bacteria, and it also enhances the uptake of the antibiotics to be effective against the Helicobacter Pylori bacteria lurking in the complex eye structures. Both antibiotics and insulin can be combined and dispensed for ophthalmic application prepared using a physiological saline solution as a major vehicle. The pH of such ophthalmic solutions should preferably be maintained between 6.5 and 7.2 with a pharmaceutically acceptable preservatives, stabilizers and surfactants as described above.

### EXAMPLE 26

The human trabecular meshwork (HTM) in POAG demonstrate an increase in extracellular matrix components and a decrease in the number of trabecular meshwork cells indicating the probable defect in the structure, function or number of trabecular meshwork cells which influences the pathogenesis of POAG. Interestingly, the pathophysiology of POAG also involves the cells of the human lamina cribrosa (HLC), which has been shown to possess a pattern of protein expression that is similar to the HTM (Steely et al. (2000) Exp Eye Res 70: 17-30).

Accordingly, POAG may have a common causal origin in the two tissues most responsible for damage to the neural retina. These causative, protein deposits and cellular decay can be corrected by using neurotophic factors along with use of topical insulin drops. Our studies show that the Insulin is a trophic factor of the nervous system.

### EXAMPLE 27

It has been found that serotonergic compounds which possess agonist activity at 5-HT receptors effectively lower and control normal and elevated IOP and are useful for treating glaucoma. This finding is further supported by our finding that the trabecular meshwork is rich in monoamine oxydase enzymes (T. R. Shantha IBID).

The anti glaucoma therapeutic agents selected from the group consisting of timolol, betaxolol, 50 levobetaxolol, carteolol, levobunolol, propranolol, brinzolamide, dorzolamide, nipradolol, Iopidine, brimonidine, pilocarpine, epinephrine, latanoprost, travoprost, unoprostone, lumigan, eliprodil and R-eliprodil, β-blockers, carbonic anhydrase inhibitors, a antagonists, α, agonists, miotics, prostaglandin analogs, hypotensive lipids, neuroprotectants. timolol, betaxolol, levobetaxolol, carteolol, levobunolol, propranolol, brinzolamide, dorzolamide, nipradolol, iopidine, brimonidine, pilocarpine, epinephrine, latanoprost, travoprost, unoprostone, lumigan, eliprodil and R-eliprodil. Insulin eye drops use along with serotonergic compounds which possess agonist activity at 5-HT, receptor sites and the other anti glaucoma therapeutic agents will help to make this invention more effective.

### EXAMPLE 28

There are Non-steroidal glucocorticoid antagonists and neurotophic growth factors are described for treating glaucoma or ocular hypertension. It is believed that the steroidal glucocorticoid antagonists bind to the glucocorticoid receptor in trabecular meshwork cells, and thereby prevent binding of endogenous glucocorticoid to the glucocorticoid receptor. They may also displace endogenous glucocorticoids bound to glucocorticoid receptors. Ketoconazole and clotrimazole are known glucocorticoid blockers.

There are numerous non-steroidal glucocorticoid antagonists with inactive ingredients. Indeed, the hypotensive steroid tetrahydrocortisol, which has been shown to lower the intraocular pressure (IOP) of glucocorticoid-induced ocular hypertension, also appears to inhibit these glucocorticoid disorders mediated changes in the HTM cytoskeleton (Clark et al (1996) Inv Ophthal 62 Vis Sci 37: 805-813).

Various exogenous growth factors have effect on trabecular meshwork. For example TGF-b isoforms significantly inhibit EGF-stimulated trabecular meshwork cell proliferation, while FGF-1, TGF-a, EGF, IL-Ia, 11-Ib, HGF, TNF-a, PDGF-AA, and IGF-1 significantly stimulated extracellular acidification (Wordinger et al. (1998) Invest Oph-thalmol Vis Sci 39: 1575-89). Specific growth factors acting through high-affinity receptors may be involved in maintaining the normal microenvironment of the trabecular meshwork and involved in pathogenesis of POAG. Any of the above selected anti glaucoma therapeutic agents including selected growth factors can be administered with insulin to enhance their therapeutic agents' effectiveness as described in our invention specially to restore or prevent the further retinal damage and loss of vision.

### EXAMPLE 29

A method of treating glaucoma by an ultrasonic device that emits ultrasonic energy, holding the ultrasonic instrument at a location external to the trabecular meshwork, transmitting the ultrasonic energy at a frequency to a desired location for a predetermined time, dislodging material built up in the trabecular meshwork, and generating heat that initiates biochemical changes in the eye to lower the IOP are described. The ultrasonic energy is frequency used ranged 20,000 to 100,000 Hz. 35 seconds. Treating with insulin drops before and after the ultrasonic method of treatment, will bring the trabecular meshwork cells physiological state and induce mitosis in these cells replacing the lost trabecular meshwork cells as seen glaucoma.

### EXAMPLE 30

The IOP-lowering agents useful in the present invention include all presently known IOP-lowering pharmaceuticals, including, but not limited to, miotics (e.g., pilocarpine, carbachol, and acetylcholinesterase inhibitors); α and α β adrenergic agonists (e.g., epinephrine, dipivalyl epinephrine, para-amino clonidine and brimonidine); betablockers (e.g., betaxolol, S-betaxolol, levobunolol, carteolol, and timolol); prostaglandins and their analogues and derivatives, such as, carbonic anhydrase inhibitors (e.g., acetazolamide, methazolamide, and ethoxzolamide, and ocular hypertensive lipids, such as those compounds (neutral replacement of the carboxylic acid group of prostaglandin F2a e.g. The preferred IOP-lowering agents are: timolol, betaxolol, S-betaxolol levobunolol, carteolol, pilocarpine, carbachol, epinephrine, dipivalyl epinephrineamethyl dipivalylepinephrine, brinzolamide, dorzolamide, unoprostone, latanoprost, travoprost, apraclonidine, and brimonidine. The selected suitable pharmacological agents from the above group are combined with insulin eye drops as described above.

Numerous modifications and alternative arrangements of steps explained herein may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form function and manner of procedure, assembly and use may be made. While the preferred embodiment of the present invention has been described, it should be understood that various changes, adaptations and modifications may be made thereto. It should be understood, therefore, that the invention is not limited to details of the illustrated invention.

While the preferred embodiment of the present invention has been described, it should be understood that various changes, adaptations and modifications may be made thereto. It should be understood, therefore, that the invention is not limited to details of the illustrated invention examples.

What is claimed is:

1. A method of lowering ocular hypertension and glaucoma, comprising the step of administering a therapeutically effective amount of insulin to an afflicted eye.

2. The method of lowering ocular hypertension and glaucoma according to claim 1 further comprising the step of applying a therapeutic agent to said afflicted eye.

3. The method of lowering ocular hypertension and glaucoma according to claim 2 wherein said therapeutic agent is a pharmaceutical agent.

4. The method of lowering ocular hypertension and glaucoma according to claim 2 wherein said therapeutic agent is a pharmaceutical biochemical agent.

5. The method of lowering ocular hypertension and glaucoma according to claim 2 wherein said known therapeutic agent is selected from a group consisting of Prostaglandin analogs comprising: Xalatan, bimatoprost Lumigan, travoprost, Travatan, Rescula and Bimatoprost.

6. The method of lowering ocular hypertension and glaucoma according to claim 2 wherein said known therapeutic agent is selected from a group consisting of Topical beta-adrenergic receptor antagonists-β blockers comprising: betaxolol, timolol, befunolol, labetalol, propranolol, bupranolol, metaprolol, bunalol, esmalol, pindolol, carteolol, hepunolol metipranolol, celiprolol, azotinolol, diacetolol, acebutolol, atenolol, Timoptic, Timoptic XE/GFS, Ocudose, Betoptic, Optipranolol, Ocupress levobunolol, Betagan, and betaxolol.

7. The method of lowering ocular hypertension and glaucoma according to claim 2 wherein said known therapeutic agent is selected from a group of Alpha2-adrenergic agonists hair growth therapeutic agents comprising: brimonidine, Alphagan, Iopidine, and Propine.

8. The method of lowering ocular hypertension and glaucoma according to claim 2 wherein said known therapeutic agent is selected from a group consisting of beta2-agonist action agents comprising: epinephrine, dipivefrin, and Propine

9. The method of lowering ocular hypertension and glaucoma according to claim 2 wherein said known therapeutic agent is selected from a group consisting of parasympathomimetic Miotic agents used in chronic glaucoma comprising: pilocarpine, and Ecothiopate.

10. The method of lowering ocular hypertension and glaucoma according to claim 2 wherein said known therapeutic agent is selected from a group consisting of Carbonic anhydrase inhibitors comprising: dorzolamide (Trusopt), brinzolamide (Azopt), acetazolamide (Diamox) and Neptazane.

11. The method of lowering the intra ocular pressure according to claim 2 wherein said known therapeutic agent is Physostigmine.

12. The method of lowering the intra ocular pressure according to claim 2 wherein said known therapeutic agent is a combination of at least two agents selected from the group comprising: Timoptic, Trusopt (Cosopt), Xalatan, Timoptic, Alphagan and Timoptic.

13. The method of lowering the intra ocular pressure according to claim 2 wherein said known therapeutic agent is extract of turmeric called curcumin.

14. The method of lowering ocular hypertension and glaucoma according to claim 2 wherein said known therapeutic agent is selected from a group consisting of NMDA-receptor antagonists comprising: ketamine, dextromethorphan, memantine, eliprodil, amantadine, methadone, dextropropoxyphene, and ketobemidone.

15. The method of lowering ocular hypertension and glaucoma according to claim 2 wherein said known therapeutic agent is selected from a group consisting of renin inhibiting compounds comprising: angiotensin II antagonist, and angiotensin converting enzyme receptor blockers.

16. The method of lowering ocular hypertension and glaucoma according to claim 3 wherein said known therapeutic agent is deprenyl.

17. The method of lowering ocular hypertension and glaucoma according to claim 2 wherein said known therapeutic agent is selected from a group comprising: Rasagiline, R(+bN-propargyl-1-aminohclan), EgLN-3, steroidal glucocorticoid antagonists, Ketoconazole and clotrimazole.

18. The method of lowering ocular hypertension and glaucoma according to claim 2 wherein said known therapeutic agent is a class I voltage-dependent ca++ channel blocking agent-a phenylalkylamine (verapamil).

19. The method of lowering ocular hypertension and glaucoma according to claim 2 further comprising the step of using an uptake facilitator to further enhance the therapeutic effect selected from the group comprising electroporation, iontophoresis, sonophoresis, vibroacoustic, vibration, heat, magnetic field, radio frequency field, microwave and light.

20. The method of lowering ocular hypertension and glaucoma according to claim 3 further comprising the step of applying an antibacterial agent to inhibit bacteria.

21. The method of lowering ocular hypertension and glaucoma according to claim 20 wherein said antibacterial agent is selected from the group consisting of amoxicillin, tetracycline, bismuth compound, erythromycin, clindamycin, metronidazole, tinidazole and quinoline compound.

22. The method of lowering ocular hypertension and glaucoma according to claim 20 further comprising the step of applying a second antibacterial agent.

23. The method of lowering ocular hypertension and glaucoma according to claim 2 wherein said therapeutic agent is a chelating agent.

24. A method of lowering ocular hypertension and glaucoma, comprising the step of administering a therapeutically effective amount of IDF-1 to an afflicted eye.

25. The method of lowering ocular hypertension and glaucoma according to claim 24 further comprising the step of applying a therapeutic agent to said afflicted eye.
